SEQUENCE LISTING (25 sequences)

SEQ ID NO: 1 - DNA, 1346 bp, Homo sapiens
ggccgccatggcggtagcgatcgctgcagcgagggtctggcggctaaaccgtggtttgagccaggctgccctcctgctgctgcggcagcctggggctcggggactggctagatctcacccccccaggcagcagcagcagttttcatctctggatgacaagccccagttcccaggggcctcggcggagtttatagataagttggaattcatccagcccaacgtcatctctggaatccccatctaccgcgtcatggaccggcaaggccagatcatcaaccccagcgaggacccccacctgccgaaggagaaggtgctgaagctctacaagagcatgacactgcttaacaccatggaccgcatcctctatgagtctcagcggcagggccggatctccttctacatgaccaactatggtgaggagggcacgcacgtggggagtgccgccgccctggacaacacggacctggtgtttggccagtaccgggaggcaggtgtgctgatgtatcgggactaccccctggaactattcatggcccagtgctatggcaacatcagtgacttgggcaaggggcgccagatgcctgtccactacggctgcaaggaacgccacttcgtcactatctcctctccactggccacgcagatccctcaggcggtgggggcggcgtacgcagccaagcgggccaatgccaacagggtcgtcatctgttacttcggcgagggggcagccagtgagggggacgcccatgccggcttcaacttcgctgccacacttgagtgccccatcatcttcttctgccggaacaatggctacgccatctccacgcccacctctgagcagtatcgcggcgatggcattgcagcacgaggccccgggtatggcatcatgtcaatccgcgtggatggtaatgatgtgtttgccgtatacaacgccacaaaggaggcccgacggcgggctgtggcagagaaccagcccttcctcatcgaggccatgacctacaggatcgggcaccacagcaccagtgacgacagttcagcgtaccgctcggtggatgaggtcaattactgggataaacaggaccaccccatctcccggctgcggcactatctgctgagccaaggctggtgggatgaggagcaggagaaggcctggaggaagcagtcccgcaggaaggtgatggaggcctttgagcaggccgagcggaagcccaaacccaaccccaacctactcttctcagacgtgtatcaggagatgcccgcccagctccgcaagcagcaggagtctctggcccgccacctgcagacctacggggagcactacccactggatcacttcgataagtgaa

SEQ ID NO: 2 - DNA, 1187 bp, Homo sapiens
ggccgccatggcggttgtagcggcggctgccggctggctactcaggctcagggcggcaggggctgaggggcactggcgtcggcttcctggcgcggggctggcgcggggctttttgcaccccgccgcgactgtcgaggatgcggcccagaggcggcaggtggctcattttactttccagccagatccggagccccgggagtacgggcaaactcagaaaatgaatcttttccagtctgtaacaagtgccttggataactcattggccaaagatcctactgcagtaatatttggtgaagatgttgcctttggtggagtctttagatgcactgttggcttgcgagacaaatatggaaaagatagagtttttaataccccattgtgtgaacaaggaattgttggatttggaatcggaattgcggtcactggagctactgccattgcggaaattcagtttgcagattatattttccctgcatttgatcagattgttaatgaagctgccaagtatcgctatcgctctggggatctttttaactgtggaagcctcactatccggtccccttggggctgtgttggtcatggggctctctatcattctcagagtcctgaagcattttttgcccattgcccaggaatcaaggtggttatacccagaagccctttccaggccaaaggacttcttttgtcatgcatagaggataaaaatccttgtatattttttgaacctaaaatactttacagggcagcagcggaagaagtccctatagaaccatacaacatcccactgtcccaggccgaagtcatacaggaagggagtgatgttactctagttgcctggggcactcaggttcatgtgatccgagaggtagcttccatggcaaaagaaaagcttggagtgtcttgtgaagtcattgatctgaggactataataccttgggatgtggacacaatttgtaagtctgtgatcaaaacagggcgactgctaatcagtcacgaggctcccttgacaggcggctttgcatcggaaatcagctctacagttcaggaggaatgtttcttgaacctagaggctcctatatcaagagtatgtggttatgacacaccatttcctcacatttttgaaccattctacatcccagacaaatggaagtgttatgatgcccttcgaaaaatgatcaactattgag

SEQ ID NO: 3 - DNA, 1346 bp, Artificial
  Synthetic Codon optimised coding sequence of human branched chain keto acid dehydrogenase E1, alpha polypeptide (BCKDHA), denominated BCKDHAco1.
ggccgccatggccgttgctatcgctgccgcgagagtatggcgacttaacaggggtctttcacaagctgctcttcttcttttgcgacagccaggcgcgcgggggcttgcccggagccatcccccccggcagcaacaacagttcagtagccttgacgataaaccgcaattcccaggcgcttcagcagagttcattgataagctggaattcattcaacccaacgtaatttccggcattcctatttatcgcgtaatggatagacaggggcaaataattaacccgagcgaggatccacatcttcccaaggaaaaagttcttaaattgtataagtctatgaccttgcttaacacgatggaccgaatactctatgaatctcagcggcagggcaggattagtttctatatgacaaattatggcgaagaaggaacccacgtcgggtccgcagcggccttggataacaccgacttggtctttggacagtaccgggaggcaggtgttcttatgtaccgggactatccccttgagctgttcatggctcaatgttatgggaacattagtgatctggggaaaggccgacaaatgcccgtgcattacggatgtaaagaaaggcattttgtaactatctcaagtcctcttgctactcaaataccgcaggccgtaggtgcggcgtatgctgctaagagggcaaacgccaatagagttgtgatatgctacttcggtgagggggctgcaagcgagggagatgcccacgccgggttcaactttgcagcgacactggagtgtcccattatatttttttgtcgaaacaatggctatgcgatctctaccccaacatcagagcagtacagaggagatgggattgcagcacggggccccggttatggaatcatgtctatacgcgtggatgggaacgacgtctttgccgtatataacgctactaaagaggccagaagacgagccgtggccgagaatcaacccttccttatagaggccatgacttacagaattggtcatcactctacgtccgatgattcttcagcttaccgcagcgtggacgaggtaaattactgggataaacaggaccatcctatttcacgacttcggcattatctcctcagccagggctggtgggacgaagaacaagaaaaggcatggagaaaacaatctagaagaaaggttatggaggcctttgagcaggcagaacgcaaaccaaaaccaaatcccaatcttcttttcagcgacgtgtaccaggaaatgccagcccagctgcggaaacagcaagaaagcctggcgagacatcttcagacctacggggaacattacccactggatcactttgacaaatgaa

SEQ ID NO: 4 - DNA, 1346 bp, Artificial
  Synthetic Codon optimised coding sequence of human branched chain keto acid dehydrogenase E1, alpha polypeptide (BCKDHA), denominated BCKDHAco2
ggccgccatggctgttgctattgctgctgcgagagtatggcgacttaacaggggtctttcacaagctgctcttcttcttttgaggcagccaggagccagagggcttgccagaagccatccccccagacagcaacaacagttcagtagccttgatgataaaccccaattcccaggagcttcagcagagttcattgataagctggaattcattcaacccaatgtaatttctggcattcctatttatagagtaatggatagacaggggcaaataattaacccctccgaggatccacatcttcccaaggaaaaagttcttaaattgtataagtctatgaccttgcttaacaccatggacaggatactctatgaatctcagagacagggcaggattagtttctatatgacaaattatggagaagaaggaacccacgtggggagcgcagccgccttggataacaccgacttggtctttggacagtacagggaggcaggtgttcttatgtacagggactatccccttgagctgttcatggctcaatgttatgggaacattagtgatctggggaaaggccgacaaatgcccgtgcattacggatgtaaagaaaggcattttgtaactatctcaagtcctcttgctactcaaataccccaggctgtaggtgccgcctatgctgctaagagggcaaacgccaatagagttgtgatatgctacttcggtgagggggctgcaagcgagggagatgcccacgctgggttcaactttgcagccacactggagtgtcccattatatttttttgtaggaacaatggctatgccatctctaccccaacatcagagcagtacagaggagatgggattgcagcaagaggccccggttatggaatcatgtctataagggtggatgggaacgacgtctttgccgtgtataacgctactaaagaggccagaagaagggctgtggctgagaatcaacccttccttatagaggccatgacttacagaattggtcatcactctacctccgatgattcttcagcttacagatccgtggatgaggtaaattactgggataaacaggaccatcctatttcaagacttaggcattatctcctcagccagggctggtgggacgaagaacaagaaaaggcatggagaaaacaatctagaagaaaggttatggaggcctttgagcaggcagaaaggaaaccaaaaccaaatcccaatcttcttttctccgacgtgtaccaggaaatgccagcccagctgaggaaacagcaagaaagcctggccagacatcttcagacctacggggaacattacccactggatcactttgacaaatgaa

SEQ ID NO: 5 - DNA, 1187 bp, Artificial
  Synthetic Codon optimised coding sequence of the human branched chain alpha-ketoacid dehydrogenase E1-beta subunit (BCKDHB), denominated BCKDHBco1
ggccgccatggcagttgtggcagccgcagcgggctggttgttgcgactcagagcagccggtgcagaaggccattggagacggttgccgggtgcgggactggcgcgcggctttctccaccccgcagcgactgtagaagacgcagcccaaagacgacaggtcgctcacttcacattccagcctgatcccgagccacgagaatacgggcaaacgcaaaaaatgaatctctttcagtccgtaacatctgctttggataatagtcttgcaaaagatccaacagctgtaattttcggggaagatgtagcgtttggcggtgtcttccgatgtaccgtcgggctgagggataagtacgggaaagatagagtatttaatacccccctgtgcgagcagggtatagtcggatttgggattggaatagccgtaacgggagcaacagcgattgccgaaatacaatttgccgactatatcttcccggcgtttgaccaaattgttaacgaggctgcgaaatatcggtatcgctccggcgacttgtttaattgcggtagcctcacaattagaagtccttgggggtgcgttggacacggtgcgctctatcacagtcaatctccagaagcttttttcgcacattgtccaggcatcaaagtagtgattccccgaagcccatttcaggcgaaaggtctcttgctctcctgtatagaagataaaaacccatgtatcttttttgagcctaaaatcctgtaccgcgccgcagctgaggaagtccctatagagccatacaacatcccactctcacaggcagaagttatacaagaagggagtgacgtgacactcgtagcatgggggacgcaggttcatgtgatcagagaggtagccagtatggcaaaagagaaattgggagtttcttgtgaagttatcgatctccgaacaataatcccttgggatgtagataccatttgtaagtctgttatcaaaactggtaggctcctcatatctcatgaggccccgttgacgggtgggttcgcgtccgaaatttcatcaactgttcaagaggagtgctttctcaacctggaagcgccgatctctagagtctgcggatatgatacccccttcccacacatatttgagcctttttatatcccggacaaatggaagtgttacgacgcccttcgaaaaatgataaattattgag

SEQ ID NO: 6 - DNA, 1187 bp, Artificial
  Synthetic Codon optimised coding sequence of the human branched chain alpha-ketoacid dehydrogenase E1-beta subunit (BCKDHB), denominated BCKDHBco2
ggccgccatggcagttgtggcagctgcagcaggctggttgttgcgcctcagagcagctggtgcagaaggccattggagaaggttgcctggtgccggactggcccgcggctttctccaccccgcagccactgtagaagatgcagcccaaagaagacaggtcgctcacttcacattccagcctgatcccgagccaagagaatatgggcaaacccaaaaaatgaatctctttcagtccgtaacatctgctttggataatagtcttgcaaaagatccaacagctgtaattttcggggaagatgtagcatttggaggtgtcttcaggtgtacagtcgggctgagggataagtacgggaaagatagagtatttaatacccccctgtgtgagcagggtatagtgggatttgggattggaatagctgtaacgggagcaacagcaattgctgaaatacaatttgctgactatatcttcccggcatttgaccaaattgttaacgaggctgcaaaatataggtataggtccggagacttgtttaattgtggtagcctcacaattagaagtccttgggggtgtgttggacatggtgcactctatcacagtcaatctccagaagcttttttcgcacattgtccaggcatcaaagtagtgattcccaggagcccatttcaggcaaaaggtctcttgctctcctgtatagaagataaaaacccatgtatcttttttgagcctaaaatcctgtacagagctgcagctgaggaagtccctatagagccatacaacatcccactctcacaggcagaagttatacaagaagggagtgatgtgacactggtagcatgggggacccaggttcatgtgatcagagaggtagccagtatggcaaaagagaaattgggagtttcttgtgaagttatcgatctccgaacaataatcccttgggatgtagataccatttgtaagtctgttatcaaaactggtaggctcctcatatctcatgaggccccgttgaccggtgggttcgcatccgaaatttcatcaactgttcaagaggagtgctttctcaacctggaagcaccaatctctagagtctgtggatatgatacccccttcccacacatatttgagcctttttatatcccagacaaatggaagtgttacgatgcccttagaaaaatgataaattattgag

SEQ ID NO: 7 - DNA, 393 bp, Homo sapiens
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 8 - DNA, 235 bp, Homo sapiens
ctagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 9 - DNA, 943 bp, Homo sapiens
gtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 10 - DNA, 542 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 11 - DNA, 208 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 12 - DNA, 130 bp, adeno-associated virus 2
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 13 - DNA, 3740 bp, Artificial
  Synthetic ITR-EF1a-BCKDHA-WT-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcaattgcgcggccgccatggcggtagcgatcgctgcagcgagggtctggcggctaaaccgtggtttgagccaggctgccctcctgctgctgcggcagcctggggctcggggactggctagatctcacccccccaggcagcagcagcagttttcatctctggatgacaagccccagttcccaggggcctcggcggagtttatagataagttggaattcatccagcccaacgtcatctctggaatccccatctaccgcgtcatggaccggcaaggccagatcatcaaccccagcgaggacccccacctgccgaaggagaaggtgctgaagctctacaagagcatgacactgcttaacaccatggaccgcatcctctatgagtctcagcggcagggccggatctccttctacatgaccaactatggtgaggagggcacgcacgtggggagtgccgccgccctggacaacacggacctggtgtttggccagtaccgggaggcaggtgtgctgatgtatcgggactaccccctggaactattcatggcccagtgctatggcaacatcagtgacttgggcaaggggcgccagatgcctgtccactacggctgcaaggaacgccacttcgtcactatctcctctccactggccacgcagatccctcaggcggtgggggcggcgtacgcagccaagcgggccaatgccaacagggtcgtcatctgttacttcggcgagggggcagccagtgagggggacgcccatgccggcttcaacttcgctgccacacttgagtgccccatcatcttcttctgccggaacaatggctacgccatctccacgcccacctctgagcagtatcgcggcgatggcattgcagcacgaggccccgggtatggcatcatgtcaatccgcgtggatggtaatgatgtgtttgccgtatacaacgccacaaaggaggcccgacggcgggctgtggcagagaaccagcccttcctcatcgaggccatgacctacaggatcgggcaccacagcaccagtgacgacagttcagcgtaccgctcggtggatgaggtcaattactgggataaacaggaccaccccatctcccggctgcggcactatctgctgagccaaggctggtgggatgaggagcaggagaaggcctggaggaagcagtcccgcaggaaggtgatggaggcctttgagcaggccgagcggaagcccaaacccaaccccaacctactcttctcagacgtgtatcaggagatgcccgcccagctccgcaagcagcaggagtctctggcccgccacctgcagacctacggggagcactacccactggatcacttcgataagtgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 3741 bp, Artificial
  Synthetic ITR-EF1a-BCKDHA-co1-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcaattgcgcggccgccatggccgttgctatcgctgccgcgagagtatggcgacttaacaggggtctttcacaagctgctcttcttcttttgcgacagccaggcgcgcgggggcttgcccggagccatcccccccggcagcaacaacagttcagtagccttgacgataaaccgcaattcccaggcgcttcagcagagttcattgataagctggaattcattcaacccaacgtaatttccggcattcctatttatcgcgtaatggatagacaggggcaaataattaacccgagcgaggatccacatcttcccaaggaaaaagttcttaaattgtataagtctatgaccttgcttaacacgatggaccgaatactctatgaatctcagcggcagggcaggattagtttctatatgacaaattatggcgaagaaggaacccacgtcgggtccgcagcggccttggataacaccgacttggtctttggacagtaccgggaggcaggtgttcttatgtaccgggactatccccttgagctgttcatggctcaatgttatgggaacattagtgatctggggaaaggccgacaaatgcccgtgcattacggatgtaaagaaaggcattttgtaactatctcaagtcctcttgctactcaaataccgcaggccgtaggtgcggcgtatgctgctaagagggcaaacgccaatagagttgtgatatgctacttcggtgagggggctgcaagcgagggagatgcccacgccgggttcaactttgcagcgacactggagtgtcccattatatttttttgtcgaaacaatggctatgcgatctctaccccaacatcagagcagtacagaggagatgggattgcagcacggggccccggttatggaatcatgtctatacgcgtggatgggaacgacgtctttgccgtatataacgctactaaagaggccagaagacgagccgtggccgagaatcaacccttccttatagaggccatgacttacagaattggtcatcactctacgtccgatgattcttcagcttaccgcagcgtggacgaggtaaattactgggataaacaggaccatcctatttcacgacttcggcattatctcctcagccagggctggtgggacgaagaacaagaaaaggcatggagaaaacaatctagaagaaaggttatggaggcctttgagcaggcagaacgcaaaccaaaaccaaatcccaatcttcttttcagcgacgtgtaccaggaaatgccagcccagctgcggaaacagcaagaaagcctggcgagacatcttcagacctacggggaacattacccactggatcactttgacaaatgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 15 - DNA, 3740 bp, Artificial
  Synthetic ITR-EF1a-BCKDHA-co2-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcaattgcgcggccgccatggctgttgctattgctgctgcgagagtatggcgacttaacaggggtctttcacaagctgctcttcttcttttgaggcagccaggagccagagggcttgccagaagccatccccccagacagcaacaacagttcagtagccttgatgataaaccccaattcccaggagcttcagcagagttcattgataagctggaattcattcaacccaatgtaatttctggcattcctatttatagagtaatggatagacaggggcaaataattaacccctccgaggatccacatcttcccaaggaaaaagttcttaaattgtataagtctatgaccttgcttaacaccatggacaggatactctatgaatctcagagacagggcaggattagtttctatatgacaaattatggagaagaaggaacccacgtggggagcgcagccgccttggataacaccgacttggtctttggacagtacagggaggcaggtgttcttatgtacagggactatccccttgagctgttcatggctcaatgttatgggaacattagtgatctggggaaaggccgacaaatgcccgtgcattacggatgtaaagaaaggcattttgtaactatctcaagtcctcttgctactcaaataccccaggctgtaggtgccgcctatgctgctaagagggcaaacgccaatagagttgtgatatgctacttcggtgagggggctgcaagcgagggagatgcccacgctgggttcaactttgcagccacactggagtgtcccattatatttttttgtaggaacaatggctatgccatctctaccccaacatcagagcagtacagaggagatgggattgcagcaagaggccccggttatggaatcatgtctataagggtggatgggaacgacgtctttgccgtgtataacgctactaaagaggccagaagaagggctgtggctgagaatcaacccttccttatagaggccatgacttacagaattggtcatcactctacctccgatgattcttcagcttacagatccgtggatgaggtaaattactgggataaacaggaccatcctatttcaagacttaggcattatctcctcagccagggctggtgggacgaagaacaagaaaaggcatggagaaaacaatctagaagaaaggttatggaggcctttgagcaggcagaaaggaaaccaaaaccaaatcccaatcttcttttctccgacgtgtaccaggaaatgccagcccagctgaggaaacagcaagaaagcctggccagacatcttcagacctacggggaacattacccactggatcactttgacaaatgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 3575 bp, Artificial
  Synthetic ITR-EF1a-BCKDHB-WT-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcaattgcgcggccgccatggcggttgtagcggcggctgccggctggctactcaggctcagggcggcaggggctgaggggcactggcgtcggcttcctggcgcggggctggcgcggggctttttgcaccccgccgcgactgtcgaggatgcggcccagaggcggcaggtggctcattttactttccagccagatccggagccccgggagtacgggcaaactcagaaaatgaatcttttccagtctgtaacaagtgccttggataactcattggccaaagatcctactgcagtaatatttggtgaagatgttgcctttggtggagtctttagatgcactgttggcttgcgagacaaatatggaaaagatagagtttttaataccccattgtgtgaacaaggaattgttggatttggaatcggaattgcggtcactggagctactgccattgcggaaattcagtttgcagattatattttccctgcatttgatcagattgttaatgaagctgccaagtatcgctatcgctctggggatctttttaactgtggaagcctcactatccggtccccttggggctgtgttggtcatggggctctctatcattctcagagtcctgaagcattttttgcccattgcccaggaatcaaggtggttatacccagaagccctttccaggccaaaggacttcttttgtcatgcatagaggataaaaatccttgtatattttttgaacctaaaatactttacagggcagcagcggaagaagtccctatagaaccatacaacatcccactgtcccaggccgaagtcatacaggaagggagtgatgttactctagttgcctggggcactcaggttcatgtgatccgagaggtagcttccatggcaaaagaaaagcttggagtgtcttgtgaagtcattgatctgaggactataataccttgggatgtggacacaatttgtaagtctgtgatcaaaacagggcgactgctaatcagtcacgaggctcccttgacaggcggctttgcatcggaaatcagctctacagttcaggaggaatgtttcttgaacctagaggctcctatatcaagagtatgtggttatgacacaccatttcctcacatttttgaaccattctacatcccagacaaatggaagtgttatgatgcccttcgaaaaatgatcaactattgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 3575 bp, Artificial
  Synthetic ITR-EF1a-BCKDHB-co1-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcaattgcgcggccgccatggcagttgtggcagccgcagcgggctggttgttgcgactcagagcagccggtgcagaaggccattggagacggttgccgggtgcgggactggcgcgcggctttctccaccccgcagcgactgtagaagacgcagcccaaagacgacaggtcgctcacttcacattccagcctgatcccgagccacgagaatacgggcaaacgcaaaaaatgaatctctttcagtccgtaacatctgctttggataatagtcttgcaaaagatccaacagctgtaattttcggggaagatgtagcgtttggcggtgtcttccgatgtaccgtcgggctgagggataagtacgggaaagatagagtatttaatacccccctgtgcgagcagggtatagtcggatttgggattggaatagccgtaacgggagcaacagcgattgccgaaatacaatttgccgactatatcttcccggcgtttgaccaaattgttaacgaggctgcgaaatatcggtatcgctccggcgacttgtttaattgcggtagcctcacaattagaagtccttgggggtgcgttggacacggtgcgctctatcacagtcaatctccagaagcttttttcgcacattgtccaggcatcaaagtagtgattccccgaagcccatttcaggcgaaaggtctcttgctctcctgtatagaagataaaaacccatgtatcttttttgagcctaaaatcctgtaccgcgccgcagctgaggaagtccctatagagccatacaacatcccactctcacaggcagaagttatacaagaagggagtgacgtgacactcgtagcatgggggacgcaggttcatgtgatcagagaggtagccagtatggcaaaagagaaattgggagtttcttgtgaagttatcgatctccgaacaataatcccttgggatgtagataccatttgtaagtctgttatcaaaactggtaggctcctcatatctcatgaggccccgttgacgggtgggttcgcgtccgaaatttcatcaactgttcaagaggagtgctttctcaacctggaagcgccgatctctagagtctgcggatatgatacccccttcccacacatatttgagcctttttatatcccggacaaatggaagtgttacgacgcccttcgaaaaatgataaattattgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 3575 bp, Artificial
  Synthetic ITR-EF1a-BCKDHB-co2-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcaattgcgcggccgccatggcagttgtggcagctgcagcaggctggttgttgcgcctcagagcagctggtgcagaaggccattggagaaggttgcctggtgccggactggcccgcggctttctccaccccgcagccactgtagaagatgcagcccaaagaagacaggtcgctcacttcacattccagcctgatcccgagccaagagaatatgggcaaacccaaaaaatgaatctctttcagtccgtaacatctgctttggataatagtcttgcaaaagatccaacagctgtaattttcggggaagatgtagcatttggaggtgtcttcaggtgtacagtcgggctgagggataagtacgggaaagatagagtatttaatacccccctgtgtgagcagggtatagtgggatttgggattggaatagctgtaacgggagcaacagcaattgctgaaatacaatttgctgactatatcttcccggcatttgaccaaattgttaacgaggctgcaaaatataggtataggtccggagacttgtttaattgtggtagcctcacaattagaagtccttgggggtgtgttggacatggtgcactctatcacagtcaatctccagaagcttttttcgcacattgtccaggcatcaaagtagtgattcccaggagcccatttcaggcaaaaggtctcttgctctcctgtatagaagataaaaacccatgtatcttttttgagcctaaaatcctgtacagagctgcagctgaggaagtccctatagagccatacaacatcccactctcacaggcagaagttatacaagaagggagtgatgtgacactggtagcatgggggacccaggttcatgtgatcagagaggtagccagtatggcaaaagagaaattgggagtttcttgtgaagttatcgatctccgaacaataatcccttgggatgtagataccatttgtaagtctgttatcaaaactggtaggctcctcatatctcatgaggccccgttgaccggtgggttcgcatccgaaatttcatcaactgttcaagaggagtgctttctcaacctggaagcaccaatctctagagtctgtggatatgatacccccttcccacacatatttgagcctttttatatcccagacaaatggaagtgttacgatgcccttagaaaaatgataaattattgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 3407 bp, Artificial
  Synthetic ITR-hAAT-BCKDHA-WT-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtaagtatcaaggttacaagacaggtttaaggagaccaatagaaactgggcttgtcgagacagagaagactcttgcgtttctgataggcacctattggtcttactgacatccactttgcctttctctccacagcaattgcgcggccgccatggcggtagcgatcgctgcagcgagggtctggcggctaaaccgtggtttgagccaggctgccctcctgctgctgcggcagcctggggctcggggactggctagatctcacccccccaggcagcagcagcagttttcatctctggatgacaagccccagttcccaggggcctcggcggagtttatagataagttggaattcatccagcccaacgtcatctctggaatccccatctaccgcgtcatggaccggcaaggccagatcatcaaccccagcgaggacccccacctgccgaaggagaaggtgctgaagctctacaagagcatgacactgcttaacaccatggaccgcatcctctatgagtctcagcggcagggccggatctccttctacatgaccaactatggtgaggagggcacgcacgtggggagtgccgccgccctggacaacacggacctggtgtttggccagtaccgggaggcaggtgtgctgatgtatcgggactaccccctggaactattcatggcccagtgctatggcaacatcagtgacttgggcaaggggcgccagatgcctgtccactacggctgcaaggaacgccacttcgtcactatctcctctccactggccacgcagatccctcaggcggtgggggcggcgtacgcagccaagcgggccaatgccaacagggtcgtcatctgttacttcggcgagggggcagccagtgagggggacgcccatgccggcttcaacttcgctgccacacttgagtgccccatcatcttcttctgccggaacaatggctacgccatctccacgcccacctctgagcagtatcgcggcgatggcattgcagcacgaggccccgggtatggcatcatgtcaatccgcgtggatggtaatgatgtgtttgccgtatacaacgccacaaaggaggcccgacggcgggctgtggcagagaaccagcccttcctcatcgaggccatgacctacaggatcgggcaccacagcaccagtgacgacagttcagcgtaccgctcggtggatgaggtcaattactgggataaacaggaccaccccatctcccggctgcggcactatctgctgagccaaggctggtgggatgaggagcaggagaaggcctggaggaagcagtcccgcaggaaggtgatggaggcctttgagcaggccgagcggaagcccaaacccaaccccaacctactcttctcagacgtgtatcaggagatgcccgcccagctccgcaagcagcaggagtctctggcccgccacctgcagacctacggggagcactacccactggatcacttcgataagtgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - DNA, 3407 bp, Artificial
  Synthetic ITR-hAAT-BCKDHA-co1-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtaagtatcaaggttacaagacaggtttaaggagaccaatagaaactgggcttgtcgagacagagaagactcttgcgtttctgataggcacctattggtcttactgacatccactttgcctttctctccacagcaattgcgcggccgccatggccgttgctatcgctgccgcgagagtatggcgacttaacaggggtctttcacaagctgctcttcttcttttgcgacagccaggcgcgcgggggcttgcccggagccatcccccccggcagcaacaacagttcagtagccttgacgataaaccgcaattcccaggcgcttcagcagagttcattgataagctggaattcattcaacccaacgtaatttccggcattcctatttatcgcgtaatggatagacaggggcaaataattaacccgagcgaggatccacatcttcccaaggaaaaagttcttaaattgtataagtctatgaccttgcttaacacgatggaccgaatactctatgaatctcagcggcagggcaggattagtttctatatgacaaattatggcgaagaaggaacccacgtcgggtccgcagcggccttggataacaccgacttggtctttggacagtaccgggaggcaggtgttcttatgtaccgggactatccccttgagctgttcatggctcaatgttatgggaacattagtgatctggggaaaggccgacaaatgcccgtgcattacggatgtaaagaaaggcattttgtaactatctcaagtcctcttgctactcaaataccgcaggccgtaggtgcggcgtatgctgctaagagggcaaacgccaatagagttgtgatatgctacttcggtgagggggctgcaagcgagggagatgcccacgccgggttcaactttgcagcgacactggagtgtcccattatatttttttgtcgaaacaatggctatgcgatctctaccccaacatcagagcagtacagaggagatgggattgcagcacggggccccggttatggaatcatgtctatacgcgtggatgggaacgacgtctttgccgtatataacgctactaaagaggccagaagacgagccgtggccgagaatcaacccttccttatagaggccatgacttacagaattggtcatcactctacgtccgatgattcttcagcttaccgcagcgtggacgaggtaaattactgggataaacaggaccatcctatttcacgacttcggcattatctcctcagccagggctggtgggacgaagaacaagaaaaggcatggagaaaacaatctagaagaaaggttatggaggcctttgagcaggcagaacgcaaaccaaaaccaaatcccaatcttcttttcagcgacgtgtaccaggaaatgccagcccagctgcggaaacagcaagaaagcctggcgagacatcttcagacctacggggaacattacccactggatcactttgacaaatgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 3407 bp, Artificial
  Synthetic ITR-hAAT-BCKDHA-co2-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtaagtatcaaggttacaagacaggtttaaggagaccaatagaaactgggcttgtcgagacagagaagactcttgcgtttctgataggcacctattggtcttactgacatccactttgcctttctctccacagcaattgcgcggccgccatggctgttgctattgctgctgcgagagtatggcgacttaacaggggtctttcacaagctgctcttcttcttttgaggcagccaggagccagagggcttgccagaagccatccccccagacagcaacaacagttcagtagccttgatgataaaccccaattcccaggagcttcagcagagttcattgataagctggaattcattcaacccaatgtaatttctggcattcctatttatagagtaatggatagacaggggcaaataattaacccctccgaggatccacatcttcccaaggaaaaagttcttaaattgtataagtctatgaccttgcttaacaccatggacaggatactctatgaatctcagagacagggcaggattagtttctatatgacaaattatggagaagaaggaacccacgtggggagcgcagccgccttggataacaccgacttggtctttggacagtacagggaggcaggtgttcttatgtacagggactatccccttgagctgttcatggctcaatgttatgggaacattagtgatctggggaaaggccgacaaatgcccgtgcattacggatgtaaagaaaggcattttgtaactatctcaagtcctcttgctactcaaataccccaggctgtaggtgccgcctatgctgctaagagggcaaacgccaatagagttgtgatatgctacttcggtgagggggctgcaagcgagggagatgcccacgctgggttcaactttgcagccacactggagtgtcccattatatttttttgtaggaacaatggctatgccatctctaccccaacatcagagcagtacagaggagatgggattgcagcaagaggccccggttatggaatcatgtctataagggtggatgggaacgacgtctttgccgtgtataacgctactaaagaggccagaagaagggctgtggctgagaatcaacccttccttatagaggccatgacttacagaattggtcatcactctacctccgatgattcttcagcttacagatccgtggatgaggtaaattactgggataaacaggaccatcctatttcaagacttaggcattatctcctcagccagggctggtgggacgaagaacaagaaaaggcatggagaaaacaatctagaagaaaggttatggaggcctttgagcaggcagaaaggaaaccaaaaccaaatcccaatcttcttttctccgacgtgtaccaggaaatgccagcccagctgaggaaacagcaagaaagcctggccagacatcttcagacctacggggaacattacccactggatcactttgacaaatgaaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 3242 bp, Artificial
  Synthetic ITR-hAAT-BCKDHB-WT-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtaagtatcaaggttacaagacaggtttaaggagaccaatagaaactgggcttgtcgagacagagaagactcttgcgtttctgataggcacctattggtcttactgacatccactttgcctttctctccacagcaattgcgcggccgccatggcggttgtagcggcggctgccggctggctactcaggctcagggcggcaggggctgaggggcactggcgtcggcttcctggcgcggggctggcgcggggctttttgcaccccgccgcgactgtcgaggatgcggcccagaggcggcaggtggctcattttactttccagccagatccggagccccgggagtacgggcaaactcagaaaatgaatcttttccagtctgtaacaagtgccttggataactcattggccaaagatcctactgcagtaatatttggtgaagatgttgcctttggtggagtctttagatgcactgttggcttgcgagacaaatatggaaaagatagagtttttaataccccattgtgtgaacaaggaattgttggatttggaatcggaattgcggtcactggagctactgccattgcggaaattcagtttgcagattatattttccctgcatttgatcagattgttaatgaagctgccaagtatcgctatcgctctggggatctttttaactgtggaagcctcactatccggtccccttggggctgtgttggtcatggggctctctatcattctcagagtcctgaagcattttttgcccattgcccaggaatcaaggtggttatacccagaagccctttccaggccaaaggacttcttttgtcatgcatagaggataaaaatccttgtatattttttgaacctaaaatactttacagggcagcagcggaagaagtccctatagaaccatacaacatcccactgtcccaggccgaagtcatacaggaagggagtgatgttactctagttgcctggggcactcaggttcatgtgatccgagaggtagcttccatggcaaaagaaaagcttggagtgtcttgtgaagtcattgatctgaggactataataccttgggatgtggacacaatttgtaagtctgtgatcaaaacagggcgactgctaatcagtcacgaggctcccttgacaggcggctttgcatcggaaatcagctctacagttcaggaggaatgtttcttgaacctagaggctcctatatcaagagtatgtggttatgacacaccatttcctcacatttttgaaccattctacatcccagacaaatggaagtgttatgatgcccttcgaaaaatgatcaactattgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 3242 bp, Artificial
  Synthetic ITR-hAAT-BCKDHB-co1-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtaagtatcaaggttacaagacaggtttaaggagaccaatagaaactgggcttgtcgagacagagaagactcttgcgtttctgataggcacctattggtcttactgacatccactttgcctttctctccacagcaattgcgcggccgccatggcagttgtggcagccgcagcgggctggttgttgcgactcagagcagccggtgcagaaggccattggagacggttgccgggtgcgggactggcgcgcggctttctccaccccgcagcgactgtagaagacgcagcccaaagacgacaggtcgctcacttcacattccagcctgatcccgagccacgagaatacgggcaaacgcaaaaaatgaatctctttcagtccgtaacatctgctttggataatagtcttgcaaaagatccaacagctgtaattttcggggaagatgtagcgtttggcggtgtcttccgatgtaccgtcgggctgagggataagtacgggaaagatagagtatttaatacccccctgtgcgagcagggtatagtcggatttgggattggaatagccgtaacgggagcaacagcgattgccgaaatacaatttgccgactatatcttcccggcgtttgaccaaattgttaacgaggctgcgaaatatcggtatcgctccggcgacttgtttaattgcggtagcctcacaattagaagtccttgggggtgcgttggacacggtgcgctctatcacagtcaatctccagaagcttttttcgcacattgtccaggcatcaaagtagtgattccccgaagcccatttcaggcgaaaggtctcttgctctcctgtatagaagataaaaacccatgtatcttttttgagcctaaaatcctgtaccgcgccgcagctgaggaagtccctatagagccatacaacatcccactctcacaggcagaagttatacaagaagggagtgacgtgacactcgtagcatgggggacgcaggttcatgtgatcagagaggtagccagtatggcaaaagagaaattgggagtttcttgtgaagttatcgatctccgaacaataatcccttgggatgtagataccatttgtaagtctgttatcaaaactggtaggctcctcatatctcatgaggccccgttgacgggtgggttcgcgtccgaaatttcatcaactgttcaagaggagtgctttctcaacctggaagcgccgatctctagagtctgcggatatgatacccccttcccacacatatttgagcctttttatatcccggacaaatggaagtgttacgacgcccttcgaaaaatgataaattattgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 3242 bp, Artificial
  Synthetic ITR-hAAT-BCKDHB-co2-ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgtaagtatcaaggttacaagacaggtttaaggagaccaatagaaactgggcttgtcgagacagagaagactcttgcgtttctgataggcacctattggtcttactgacatccactttgcctttctctccacagcaattgcgcggccgccatggcagttgtggcagctgcagcaggctggttgttgcgcctcagagcagctggtgcagaaggccattggagaaggttgcctggtgccggactggcccgcggctttctccaccccgcagccactgtagaagatgcagcccaaagaagacaggtcgctcacttcacattccagcctgatcccgagccaagagaatatgggcaaacccaaaaaatgaatctctttcagtccgtaacatctgctttggataatagtcttgcaaaagatccaacagctgtaattttcggggaagatgtagcatttggaggtgtcttcaggtgtacagtcgggctgagggataagtacgggaaagatagagtatttaatacccccctgtgtgagcagggtatagtgggatttgggattggaatagctgtaacgggagcaacagcaattgctgaaatacaatttgctgactatatcttcccggcatttgaccaaattgttaacgaggctgcaaaatataggtataggtccggagacttgtttaattgtggtagcctcacaattagaagtccttgggggtgtgttggacatggtgcactctatcacagtcaatctccagaagcttttttcgcacattgtccaggcatcaaagtagtgattcccaggagcccatttcaggcaaaaggtctcttgctctcctgtatagaagataaaaacccatgtatcttttttgagcctaaaatcctgtacagagctgcagctgaggaagtccctatagagccatacaacatcccactctcacaggcagaagttatacaagaagggagtgatgtgacactggtagcatgggggacccaggttcatgtgatcagagaggtagccagtatggcaaaagagaaattgggagtttcttgtgaagttatcgatctccgaacaataatcccttgggatgtagataccatttgtaagtctgttatcaaaactggtaggctcctcatatctcatgaggccccgttgaccggtgggttcgcatccgaaatttcatcaactgttcaagaggagtgctttctcaacctggaagcaccaatctctagagtctgtggatatgatacccccttcccacacatatttgagcctttttatatcccagacaaatggaagtgttacgatgcccttagaaaaatgataaattattgaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

(19) United States
(12) Patent Application Publication
(10) Pub. No.: US 2023/0136699 A1
(43) Pub. Date: May 4, 2023

(54) GENE THERAPY FOR MAPLE SYRUP URINE DISEASE

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR)

(72) Inventors: Manuel SCHIFF, Paris (FR); Marina CAVAZZANA, Paris (FR); Pascale DE LONLAY-DEBENEY, Paris (FR); Marcelo SIMON SOLA, Paris (FR); Clément PONTOIZEAU, Paris (FR); Chris OTTOLENGHI, Paris (FR)

(21) Appl. No.: 17/904,124

(22) PCT Filed: Feb. 26, 2021
(86) PCT No.: PCT/EP2021/054797

§ 371 Date: Aug. 12, 2022

(30) Foreign Application Priority Data
Feb. 28, 2020 (EP) 20305201.4

(51) Int. Cl. C12N 9/02; C12N 15/86; A61P 3/00; A61P 13/00; A61K 38/44

(52) U.S. Cl. CPC C12N 9/0008; C12Y 102/04004; C12N 15/86; A61P 3/00; A61P 13/00; A61K 38/44; C12N 2750/14143; C12N 2830/50; C12N 2750/14171

(57) ABSTRACT

Maple syrup urine disease (MSUD) is a rare autosomal recessive disease with an incidence that is caused by a defective activity of the branched-chain 2-keto acid dehydrogenase (BCKD) leading to accumulation of branched-chain amino acids (BCAA) leucine, isoleucine, valine and their corresponding alpha-ketoacids (BCKA) in tissues and body fluids. The inventors herein characterized the Bckdha^{-/-} mouse and Bckdhb^{-/-} mouse recapitulating the classical forms of MSUD. As a proof of concept, they developed a (liver-directed) AAV gene therapy based on the transfer of human BCKDHA (hBCKDHA) or BCKDHB (hBCKDHB) mediated by AAV8 during immediate neonatal period in Bckdha−/− or Bckdhb^{-/-} mice. The inventors demonstrated that hBCKDHA gene transfer completely rescued the lethal early-onset phenotype of Bckdha−/− mice allowing long-term survival to age 12 months, at which they were systematically sacrificed, without overt phenotypic abnormalities. They also demonstrated that hBCKDHB gene transfer exhibited similar survival and a normal growth without overt phenotypic abnormalities at age 3 months, with a dramatic improvement of the biochemical phenotype. The present invention relates to a method of treating MSUD by gene therapy.

### FIELD OF THE INVENTION

The present invention is in the field of medicine, in particular in rare diseases.

### BACKGROUND OF THE INVENTION

Maple syrup urine disease (MSUD, MIM: 248600) is a rare autosomal recessive disease with an incidence of one in 185,000 live births. This disorder is caused by a defective activity of the branched-chain 2-keto acid dehydrogenase (BCKD) leading to accumulation of branched-chain amino acids (BCAA) leucine, isoleucine, valine and their corresponding alpha-ketoacids (BCKA) in tissues and body fluids (Strauss et al., 2013). The BCKD enzyme is a multi-enzyme complex with four components, branched-chain keto acid decarboxylase alpha and beta subunits (E1_{α} and E1_{β}), dihydrolipoyl transacylase (E2) subunit and dihydrolipoamide dehydrogenase (E3) subunit. MSUD is due to mutations in BCKDHA, BCKDHB and DBT genes respectively coding for E1_{α}, E1_{β} and E2 subunits and accounting for 45%, 35% and 20% of MSUD patients respectively (Strauss et al., 2013). Neurotoxicity in MSUD was shown to be related to accumulation of leucine and α-ketoisocaproic acid (αKIC, the ketoacid derived from leucine) (Muelly et al., 2013). In the classical severe form of MSUD, with less than 3% residual enzyme activity, this accumulation causes coma and cerebral edema shortly after birth with early death in the absence of appropriate management.

MSUD represents an unmet clinical need. Current MSUD treatment is limited to very severe and life-long BCAA dietary restriction associated with an oral BCAA-free amino acids mixture. Such treatment is difficult to maintain on the long-term, largely incompatible with a normal professional life. Further, it does not prevent long-term neurocognitive (Bouchereau et al., 2017) and psychiatric issues (Abi-Wardé et al., 2017). Orthotopic liver transplantation (OLT), was shown to be an effective therapy for MSUD allowing removal of dietary restrictions, complete protection from acute decompensations during illness (Bodner-Leidecker et al., 2000; Wendel et al., 1999), arrest (although not reversion) of neurocognitive impairment progression (Mazariegos et al., 2012; Muelly et al., 2013), prevention of life-threatening cerebral edema (Muelly et al., 2013), metabolic and clinical stability (Mazariegos et al., 2012). However OLT is a therapeutic option available only for a few patients and is associated with the potential (though low) risk of death and graft failures (Mazariegos et al., 2012). As a monogenic disease MSUD represents an ideal target for liver-directed gene therapy since clinical OLT data suggests that the restoration of liver BCKD enzyme activity (only contributing to 9-13% of whole-body BCKD activity (Suryawan et al., 1998)) is fully therapeutic. This was an incentive to test liver gene transfer in an MSUD mouse model.

Among the available gene-delivery vehicles, adeno-associated virus (AAV) vectors are the most suitable for liver gene transfer. AAV liver gene therapy achieved a major milestone with the proof of safety and long-term efficacy in a clinical trial for haemophilia B (Nathwani et al., 2014). Inborn errors of metabolism are good candidates for AAV gene therapy (Ginocchio et al., 2019). Proofs of concept of efficacy were obtained in mice for urea cycle disorders (Baruteau et al., 2018; Chandler et al., 2013; Cunningham et al., 2009; Lee et al., 2012), organic acidemias (Chandler and Venditti, 2019) or phenylketonuria (Grisch-Chan et al., 2019) and human clinical trials are currently being conducted for ornithine transcarbamylase deficiency (OTC) (NCT02991144), glycogen storage disease type 1a (NCT03517085), mucopolysaccharidosis type VI (MPSVI) (NCT03173521) and Pompe disease (NCT03533673).

Mouse models of MSUD with mutations in the Dbt gene have been developed and characterized (Homanics et al., 2006; S Sonnet et al., 2016; Zinnanti et al., 2009). While the majority of patients harbour mutations in BCKDHA and BCKDHB genes, there is, to our knowledge, no characterised mouse model of MSUD involving the Bckdha or Bckdhb genes. Recently, a mouse model with tissue-specific Bckdha knockout in brown adipose tissue was described and showed a reduced tolerance of BCAA loading but no other phenotypic features of MSUD (Yoneshiro et al., 2019).

### SUMMARY OF THE INVENTION

As defined by the claims, the present invention relates to a method of treating Maple syrup urine disease (MSUD) by gene therapy.

### DETAILED DESCRIPTION OF THE INVENTION

The inventors herein characterized the Bckdha−/− mouse, recapitulating the classical form of MSUD. As a proof of concept, they developed a (liver-directed) AAV gene therapy based on the transfer of human BCKDHA (hBCKDHA) mediated by AAV8 during immediate neonatal period in Bckdha−/− mice. The inventors demonstrated that hBCKDHA gene transfer completely rescued the lethal early-onset phenotype of Bckdha−/− mice allowing long-term survival to 12 months without overt phenotypic abnormalities. Mice were systematically sacrificed at the age of 12 months.

The first object of the present invention relates to a recombinant nucleic acid molecule comprising a transgene encoding for the branched-chain keto acid decarboxylase alpha or beta subunit wherein the transgene is operatively linked to a promoter.

As used herein, the term “nucleic acid molecule” has its general meaning in the art and refers to a DNA molecule.

As used herein, the term “transgene” refers to any nucleic acid that shall be expressed in a mammal cell.

In some embodiments, the transgene comprises a nucleic acid sequence having at least 80% of identity with SEQ ID NO:1 or SEQ ID NO:2.

> Coding sequence of human branched
chain keto acid dehydrogenase El,
alpha polypeptide (BCKDHA),
denominated BCKDHA CDS WT.
SEQ ID NO: 1
ggccgccatggcggtagcgatcgctgcagcgagggtctgg
cggctaaaccgtggtttgagccaggctgccctcctgctgc
tgcggcagcctggggctcggggactggctagatctcaccc
ccccaggcagcagcagcagttttcatctctggatgacaag
ccccagttcccaggggcctcggcggagtttatagataagt
tggaattcatccagcccaacgtcatctctggaatccccat
ctaccgcgtcatggaccggcaaggccagatcatcaacccc
agcgaggacccccacctgccgaaggagaaggtgctgaagc
tctacaagagcatgacactgcttaacaccatggaccgcat
cctctatgagtctcagcggcagggccggatctccttctac
atgaccaactatggtgaggagggcacgcacgtggggagtg
ccgccgccctggacaacacggacctggtgtttggccagta
ccgggaggcaggtgtgctgatgtatcgggactaccccctg
gaactattcatggcccagtgctatggcaacatcagtgact
tgggcaaggggcgccagatgcctgtccactacggctgcaa
ggaacgccacttcgtcactatctcctctccactggccacg
cagatccctcaggcggtgggggcggcgtacgcagccaagc
gggccaatgccaacagggtcgtcatctgttacttcggcga
gggggcagccagtgagggggacgcccatgccggcttcaac
ttcgctgccacacttgagtgccccatcatcttcttctgcc
ggaacaatggctacgccatctccacgcccacctctgagca
gtatcgcggcgatggcattgcagcacgaggccccgggtat
ggcatcatgtcaatccgcgtggatggtaatgatgtgtttg
ccgtatacaacgccacaaaggaggcccgacggcgggctgt
ggcagagaaccagcccttcctcatcgaggccatgacctac
aggatcgggcaccacagcaccagtgacgacagttcagcgt
accgctcggtggatgaggtcaattactgggataaacagga
ccaccccatctcccggctgcggcactatctgctgagccaa
ggctggtgggatgaggagcaggagaaggcctggaggaagc
agtcccgcaggaaggtgatggaggcctttgagcaggccga
gcggaagcccaaacccaaccccaacctactcttctcagac
gtgtatcaggagatgcccgcccagctccgcaagcagcagg
agtctctggcccgccacctgcagacctacggggagcacta
cccactggatcacttcgataagtgaa
> Coding sequence of human branched
chain alpha-keto acid dehydrogenase
El-beta subunit (BCKDHB), denominated
BCKDHB CDS WT.
SEQ ID NO: 2
ggccgccatggcggttgtagcggcggctgccggctggcta
ctcaggctcagggcggcaggggctgaggggcactggcgtc
ggcttcctggcgcggggctggcgcggggctttttgcaccc
cgccgcgactgtcgaggatgcggcccagaggcggcaggtg
gctcattttactttccagccagatccggagccccgggagt
acgggcaaactcagaaaatgaatcttttccagtctgtaac
aagtgccttggataactcattggccaaagatcctactgca
gtaatatttggtgaagatgttgcctttggtggagtcttta
gatgcactgttggcttgcgagacaaatatggaaaagatag
agtttttaataccccattgtgtgaacaaggaattgttgga
tttggaatcggaattgcggtcactggagctactgccattg
cggaaattcagtttgcagattatattttccctgcatttga
tcagattgttaatgaagctgccaagtatcgctatcgctct
ggggatctttttaactgtggaagcctcactatccggtccc
cttggggctgtgttggtcatggggctctctatcattctca
gagtcctgaagcattttttgcccattgcccaggaatcaag
gtggttatacccagaagccctttccaggccaaaggacttc
ttttgtcatgcatagaggataaaaatccttgtatattttt
tgaacctaaaatactttacagggcagcagcggaagaagtc
cctatagaaccatacaacatcccactgtcccaggccgaag
tcatacaggaagggagtgatgttactctagttgcctgggg
cactcaggttcatgtgatccgagaggtagcttccatggca
aaagaaaagcttggagtgtcttgtgaagtcattgatctga
ggactataataccttgggatgtggacacaatttgtaagtc
tgtgatcaaaacagggcgactgctaatcagtcacgaggct
cccttgacaggcggctttgcatcggaaatcagctctacag
ttcaggaggaatgtttcttgaacctagaggctcctatatc
aagagtatgtggttatgacacaccatttcctcacattttt
gaaccattctacatcccagacaaatggaagtgttatgatg
cccttcgaaaaatgatcaactattgag

According to the invention a first nucleic acid sequence having at least 80% of identity with a second nucleic acid sequence means that the first sequence has 80, 81, 82, 83, 84, 85, 86, 87, 88, 89, 90; 91; 92; 93; 94; 95; 96; 97; 98; 99 or 100% of identity with the second nucleic acid sequence.

As used herein, the term “sequence identity,” as used herein, has the standard meaning in the art. As is known in the art, a number of different programs can be used to identify whether a nucleic acid sequence has sequence identity or similarity to another nucleic acid sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387 (1984), preferably using the default settings, or by inspection. An example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

In some embodiments, the sequence of the transgene is codon-optimized. As used herein, the term “codon-optimized” refers to nucleic sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell.

In some embodiments, the transgene comprises the nucleic acid sequence of SEQ ID NO:3 or SEQ ID NO:4.

> Codon optimised coding sequence of
human branched chain keto acid
dehydrogenase El, alpha polypeptide
(BCKDHA), denominated BCKDHAco1.
SEQ ID NO: 3
ggccgccatggccgttgctatcgctgccgcgagagtatgg
cgacttaacaggggtctttcacaagctgctcttcttcttt
tgcgacagccaggcgcgcgggggcttgcccggagccatcc
cccccggcagcaacaacagttcagtagccttgacgataaa
ccgcaattcccaggcgcttcagcagagttcattgataagc
tggaattcattcaacccaacgtaatttccggcattcctat
ttatcgcgtaatggatagacaggggcaaataattaacccg
agcgaggatccacatcttcccaaggaaaaagttcttaaat
tgtataagtctatgaccttgcttaacacgatggaccgaat
actctatgaatctcagcggcagggcaggattagtttctat
atgacaaattatggcgaagaaggaacccacgtcgggtccg
cagcggccttggataacaccgacttggtctttggacagta
ccgggaggcaggtgttcttatgtaccgggactatcccctt
gagctgttcatggctcaatgttatgggaacattagtgatc
tggggaaaggccgacaaatgcccgtgcattacggatgtaa
agaaaggcattttgtaactatctcaagtcctcttgctact
caaataccgcaggccgtaggtgcggcgtatgctgctaaga
gggcaaacgccaatagagttgtgatatgctacttcggtga
gggggctgcaagcgagggagatgcccacgccgggttcaac
tttgcagcgacactggagtgtcccattatatttttttgtc
gaaacaatggctatgcgatctctaccccaacatcagagca
gtacagaggagatgggattgcagcacggggccccggttat
ggaatcatgtctatacgcgtggatgggaacgacgtctttg
ccgtatataacgctactaaagaggccagaagacgagccgt
ggccgagaatcaacccttccttatagaggccatgacttac
agaattggtcatcactctacgtccgatgattcttcagctt
accgcagcgtggacgaggtaaattactgggataaacagga
ccatcctatttcacgacttcggcattatctcctcagccag
ggctggtgggacgaagaacaagaaaaggcatggagaaaac
aatctagaagaaaggttatggaggcctttgagcaggcaga
acgcaaaccaaaaccaaatcccaatcttcttttcagcgac
gtgtaccaggaaatgccagcccagctgcggaaacagcaag
aaagcctggcgagacatcttcagacctacggggaacatta
cccactggatcactttgacaaatgaa
> Codon optimised coding sequence of human
branched chain keto acid dehydrogenase El,
alpha polypeptide (BCKDHA), denominated
BCKDHAco2.
SEQ ID NO: 4
ggccgccatggctgttgctattgctgctgcgagagtatgg
cgacttaacaggggtctttcacaagctgctcttcttcttt
tgaggcagccaggagccagagggcttgccagaagccatcc
ccccagacagcaacaacagttcagtagccttgatgataaa
ccccaattcccaggagcttcagcagagttcattgataagc
tggaattcattcaacccaatgtaatttctggcattcctat
ttatagagtaatggatagacaggggcaaataattaacccc
tccgaggatccacatcttcccaaggaaaaagttcttaaat
tgtataagtctatgaccttgcttaacaccatggacaggat
actctatgaatctcagagacagggcaggattagtttctat
atgacaaattatggagaagaaggaacccacgtggggagcg
cagccgccttggataacaccgacttggtctttggacagta
cagggaggcaggtgttcttatgtacagggactatcccctt
gagctgttcatggctcaatgttatgggaacattagtgatc
tggggaaaggccgacaaatgcccgtgcattacggatgtaa
agaaaggcattttgtaactatctcaagtcctcttgctact
caaataccccaggctgtaggtgccgcctatgctgctaaga
gggcaaacgccaatagagttgtgatatgctacttcggtga
gggggctgcaagcgagggagatgcccacgctgggttcaac
tttgcagccacactggagtgtcccattatatttttttgta
ggaacaatggctatgccatctctaccccaacatcagagca
gtacagaggagatgggattgcagcaagaggccccggttat
ggaatcatgtctataagggtggatgggaacgacgtctttg
ccgtgtataacgctactaaagaggccagaagaagggctgt
ggctgagaatcaacccttccttatagaggccatgacttac
agaattggtcatcactctacctccgatgattcttcagctt
acagatccgtggatgaggtaaattactgggataaacagga
ccatcctatttcaagacttaggcattatctcctcagccag
ggctggtgggacgaagaacaagaaaaggcatggagaaaac
aatctagaagaaaggttatggaggcctttgagcaggcaga
aaggaaaccaaaaccaaatcccaatcttcttttctccgac
gtgtaccaggaaatgccagcccagctgaggaaacagcaag
aaagcctggccagacatcttcagacctacggggaacatta
cccactggatcactttgacaaatgaa

In some embodiments, the transgene comprises the nucleic acid sequence of SEQ ID NO:5 or SEQ ID NO:6.

SEQ ID NO: 5
> Codon optimised coding sequence of the human
branched chain alpha-ketoacid dehydrogenase E1-
beta subunit(BCKDHB), denominated BCKDHBco1.
ggccgccatggcagttgtggcagccgcagcgggctggttgttgcgactca
gagcagccggtgcagaaggccattggagacggttgccgggtgcgggactg
gcgcgcggctttctccaccccgcagcgactgtagaagacgcagcccaaag
acgacaggtcgctcacttcacattccagcctgatcccgagccacgagaat
acgggcaaacgcaaaaaatgaatctctttcagtccgtaacatctgctttg
gataatagtcttgcaaaagatccaacagctgtaattttcggggaagatgt
agcgtttggcggtgtcttccgatgtaccgtcgggctgagggataagtacg
ggaaagatagagtatttaatacccccctgtgcgagcagggtatagtcgga
tttgggattggaatagccgtaacgggagcaacagcgattgccgaaataca
atttgccgactatatcttcccggcgtttgaccaaattgttaacgaggctg
cgaaatatcggtatcgctccggcgacttgtttaattgcggtagcctcaca
attagaagtccttgggggtgcgttggacacggtgcgctctatcacagtca
atctccagaagcttttttcgcacattgtccaggcatcaaagtagtgattc
cccgaagcccatttcaggcgaaaggtctcttgctctcctgtatagaagat
aaaaacccatgtatcttttttgagcctaaaatcctgtaccgcgccgcagc
tgaggaagtccctatagagccatacaacatcccactctcacaggcagaag
ttatacaagaagggagtgacgtgacactcgtagcatgggggacgcaggtt
catgtgatcagagaggtagccagtatggcaaaagagaaattgggagtttc
ttgtgaagttatcgatctccgaacaataatcccttgggatgtagatacca
tttgtaagtctgttatcaaaactggtaggctcctcatatctcatgaggcc
ccgttgacgggtgggttcgcgtccgaaatttcatcaactgttcaagagga
gtgctttctcaacctggaagcgccgatctctagagtctgcggatatgata
cccccttcccacacatatttgagcctttttatatcccggacaaatggaag
tgttacgacgcccttcgaaaaatgataaattattgag
SEQ ID NO: 6
> Codon optimised coding sequence of the human
branched chain alpha-ketoacid dehydrogenase E1-
beta subunit(BCKDHB), denominated BCKDHBco2.
ggccgccatggcagttgtggcagctgcagcaggctggttgttgcgcctca
gagcagctggtgcagaaggccattggagaaggttgcctggtgccggactg
gcccgcggctttctccaccccgcagccactgtagaagatgcagcccaaag
aagacaggtcgctcacttcacattccagcctgatcccgagccaagagaat
atgggcaaacccaaaaaatgaatctctttcagtccgtaacatctgctttg
gataatagtcttgcaaaagatccaacagctgtaattttcggggaagatgt
agcatttggaggtgtcttcaggtgtacagtcgggctgagggataagtacg
ggaaagatagagtatttaatacccccctgtgtgagcagggtatagtggga
tttgggattggaatagctgtaacgggagcaacagcaattgctgaaataca
atttgctgactatatcttcccggcatttgaccaaattgttaacgaggctg
caaaatataggtataggtccggagacttgtttaattgtggtagcctcaca
attagaagtccttgggggtgtgttggacatggtgcactctatcacagtca
atctccagaagcttttttcgcacattgtccaggcatcaaagtagtgattc
ccaggagcccatttcaggcaaaaggtctcttgctctcctgtatagaagat
aaaaacccatgtatcttttttgagcctaaaatcctgtacagagctgcagc
tgaggaagtccctatagagccatacaacatcccactctcacaggcagaag
ttatacaagaagggagtgatgtgacactggtagcatgggggacccaggtt
catgtgatcagagaggtagccagtatggcaaaagagaaattgggagtttc
ttgtgaagttatcgatctccgaacaataatcccttgggatgtagatacca
tttgtaagtctgttatcaaaactggtaggctcctcatatctcatgaggcc
ccgttgaccggtgggttcgcatccgaaatttcatcaactgttcaagagga
gtgctttctcaacctggaagcaccaatctctagagtctgtggatatgata
cccccttcccacacatatttgagcctttttatatcccagacaaatggaag
tgttacgatgcccttagaaaaatgataaattattgag

As used herein, the terms “promoter” has its general meaning in the art and refers to a segment of a nucleic acid sequence, typically but not limited to DNA that controls the transcription of the nucleic acid sequence to which it is operatively linked. The promoter region includes specific sequences that are sufficient for RNA polymerase recognition, binding and transcription initiation. In addition, the promoter region can optionally include sequences which modulate this recognition, binding and transcription initiation activity of RNA polymerase. The skilled person will be aware that promoters are built from stretches of nucleic acid sequences and often comprise elements or functional units in those stretches of nucleic acid sequences, such as a transcription start site, a binding site for RNA polymerase, general transcription factor binding sites, such as a TATA box, specific transcription factor binding sites, and the like. Further regulatory sequences may be present as well, such as enhancers, and sometimes introns at the end of a promoter sequence.

Typically, the promoter may be an ubiquitous or tissue-specific promoter, in particular a promoter able to promote expression in cells or tissues in which expression of the transgene is desirable such as in cells or tissues in which the transgene expression is desirable.

In some embodiments, the promoter is a liver-specific promoter such as the alpha-1 antitrypsin promoter (hAAT), the transthyretin promoter, the albumin promoter, the thyroxine-binding globulin (TBG) promoter, the LSP promoter (comprising a thyroid hormone-binding globulin promoter sequence, two copies of an alphal-microglobulin/bikunin enhancer sequence, and a leader sequence—34.111, C. R., et al. (1997). Optimization of the human factor VIII complementary DNA expression plasmid for gene therapy of hemophilia A. Blood Coag. Fibrinol. 8: S23-S30.), etc. Other useful liver-specific promoters are known in the art, for example those listed in the Liver Specific Gene Promoter Database compiled the Cold Spring Harbor Laboratory (http://rulai.cshl.edu/LSPD/).

In some embodiments, the promoter is the hAAT promoter. As used herein, the term “hAAT” as its general meaning in the art and refers to the promoter of the gene encoding for the human alpha 1-antitrypsin.

In some embodiments, the hAAT promoter comprises the nucleic acid sequence of SEQ ID NO:7.

SEQ ID NO: 7
> promoter of the gene encoding for human alpha 1-
antitrypsin
gatcttgctaccagtggaacagccactaaggattctgcagtgagagcaga
gggccagctaagtggtactctcccagagactgtctgactcacgccacccc
ctccaccttggacacaggacgctgtggtttctgagccaggtacaatgact
cctttcggtaagtgcagtggaagctgtacactgcccaggcaaagcgtccg
ggcagcgtaggcgggcgactcagatcccagccagtggacttagcccctgt
ttgctcctccgataactggggtgaccttggttaatattcaccagcagcct
cccccgttgcccctctggatccactgcttaaatacggacgaggacagggc
cctgtctcctcagcttcaggcaccaccactgacctgggacagt

Other tissue-specific or non-tissue-specific promoters may be useful in the practice of the invention.

In some embodiments, the promoter is a ubiquitous promoter. Representative ubiquitous promoters include the cytomegalovirus enhancer/chicken beta actin (CAG) promoter, the cytomegalovirus enhancer/promoter (CMV), the PGK promoter, the SV40 early promoter, etc.

In some embodiments, the promoter is the EF1a promoter. As used herein, the term “EF1a promoter” has its general meaning in the art and refers to the promoter of the gene encoding for elongation factor-1 alpha.

In some embodiments, the EF1a promoter comprises the nucleic acid sequence of SEQ ID NO:8.

SEQ ID NO: 8
> promoter of the gene encoding for elongation
factor-1 alpha
ctagcgctccggtgcccgtcagtgggcagagcgcacatcgcccacagtcc
ccgagaagttggggggaggggtcggcaattgaaccggtgcctagagaagg
tggcgcggggtaaactgggaaagtgatgtcgtgtactggctccgcctttt
tcccgagggtgggggagaaccgtatataagtgcagtagtcgccgtgaacg
ttctttttcgcaacgggtttgccgccagaacacag

In some embodiments, the EF1a promoter of the present invention further comprises an extra intronic sequence that will increase the expression of the transgene by the promoter. Typically, said extra intronic sequence consists of the nucleic acid sequence of SEQ ID NO:9.

SEQ ID NO: 9
> This extra intronic sequence gives a higher
level of expression than the core EF1a promoter
alone.
gtaagtgccgtgtgtggttcccgcgggcctggcctctttacgggttatgg
cccttgcgtgccttgaattacttccacgcccctggctgcagtacgtgatt
cttgatcccgagcttcgggttggaagtgggtgggagagttcgaggccttg
cgcttaaggagccccttcgcctcgtgcttgagttgaggcctggcctgggc
gctggggccgccgcgtgcgaatctggtggcaccttcgcgcctgtctcgct
gctttcgataagtctctagccatttaaaatttttgatgacctgctgcgac
gctttttttctggcaagatagtcttgtaaatgcgggccaagatctgcaca
ctggtatttcggtttttggggccgcgggcggcgacggggcccgtgcgtcc
cagcgcacatgttcggcgaggcggggcctgcgagcgcggccaccgagaat
cggacgggggtagtctcaagctggccggcctgctctggtgcctggcctcg
cgccgccgtgtatcgccccgccctgggcggcaaggctggcccggtcggca
ccagttgcgtgagcggaaagatggccgcttcccggccctgctgcagggag
ctcaaaatggaggacgcggcgctcgggagagcgggcgggtgagtcaccca
cacaaaggaaaagggcctttccgtcctcagccgtcgcttcatgtgactcc
acggagtaccgggcgccgtccaggcacctcgattagttctcgagcttttg
gagtacgtcgtctttaggttggggggaggggttttatgcgatggagtttc
cccacactgagtgggtggagactgaagttaggccagcttggcacttgatg
taattctccttggaatttgccctttttgagtttggatcttggttcattct
caagcctcagacagtggttcaaagtttttttcttccatttcag

As used herein, the terms “operably linked”, or “operatively linked” are used interchangeably herein, and refer to the functional relationship of the nucleic acid sequences with regulatory sequences of nucleotides, such as promoters, enhancers, transcriptional and translational stop sites, and other signal sequences and indicates that two or more DNA segments are joined together such that they function in concert for their intended purposes. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

In some embodiments, further regulatory sequences may also be added to the recombinant nucleic acid molecule of the present invention.

As used herein, the term “regulatory sequence” is used interchangeably with “regulatory element” herein and refers to a segment of nucleic acid, typically but not limited to DNA, that modulate the transcription of the nucleic acid sequence to which it is operatively linked, and thus acts as a transcriptional modulator. A regulatory sequence often comprises nucleic acid sequences that are transcription binding domains that are recognized by the nucleic acid-binding domains of transcriptional proteins and/or transcription factors, enhancers or repressors etc.

In some embodiments, the nucleic acid molecule of the present invention comprises a Woodchuck Hepatitis Virus (WHP) Posttranscriptional Regulatory Element (WPRE) sequence that is a DNA sequence that, when transcribed creates a tertiary structure enhancing expression, by stabilization of the messenger RNA. Typically, the WPRE sequence is inserted downstream to the transgene. In some embodiments, the recombinant acid molecule of the present invention comprises a WPRE sequence devoid of X protein open reading frames (ORFs), that allows to remove oncogenic side effect without significant loss of RNA enhancement activity (Schambach, A. et al. Woodchuck hepatitis virus post-transcriptional regulatory element deleted from X protein and promoter sequences enhances retroviral vector titer and expression. Gene Ther. 13, 641-645 (2006)).

In some embodiments, the WPRE sequence comprises the nucleic acid sequence of SEQ ID NO: 10.

SEQ ID NO: 10
> Woodchuck hepatitis virus post-transcriptional
regulatory element (WPRE) is a sequence that
stimulates the expression of transgenes via
increased nuclear export.
aatcaacctctggattacaaaatttgtgaaagattgactggtattcttaa
ctatgttgctccttttacgctatgtggatacgctgctttaatgcctttgt
atcatgctattgcttcccgtatggctttcattttctcctccttgtataaa
tcctggttgctgtctctttatgaggagttgtggcccgttgtcaggcaacg
tggcgtggtgtgcactgtgtttgctgacgcaacccccactggttggggca
ttgccaccacctgtcagctcctttccgggactttcgctttccccctccct
attgccacggcggaactcatcgccgcctgccttgcccgctgctggacagg
ggctcggctgttgggcactgacaattccgtggtgttgtcggggaaatcat
cgtcctttccttggctgctcgcctgtgttgccacctggattctgcgcggg
acgtccttctgctacgtcccttcggccctcaatccagcggaccttccttc
ccgcggcctgctgccggctctgcggcctcttccgcgtcttcg

In some embodiments, the recombinant nucleic acid molecule of the present invention comprises a polyadenylation signal sequence inserted downstream to the transgene.

As used herein, the term “polyadenylation signal sequence” has its general meaning in the art and refers to a nucleic acid sequence that mediates the attachment of a polyadenine stretch to the 3′ terminus of the mRNA. Suitable polyadenylation signals include the SV40 early polyadenylation signal, the SV40 late polyadenylation signal, the HSV thymidine kinase polyadenylation signal, the protamine gene polyadenylation signal, the adenovirus 5 EIb polyadenylation signal, the bovine growth hormone polyadenylation signal, the human variant growth hormone polyadenylation signal and the like.

In some embodiments, the polyadenylation sequence comprises the nucleic acid sequence of SEQ ID NO: 11.

SEQ ID NO: 11
> Bovine growth hormone polyA signal is a termi-
nator it's role is to define the end of a
transcriptional unit (such as a gene) and
initiate the process of releasing the newly
synthesized RNA from the transcription machinery.
ctgtgccttctagttgccagccatctgttgtttgcccctcccccgtgcct
tccttgaccctggaaggtgccactcccactgtcctttcctaataaaatga
ggaaattgcatcgcattgtctgagtaggtgtcattctattctggggggtg
gggtggggcaggacagcaagggggaggattgggaagacaatagcaggcat
gctgggga

In some embodiments, the recombinant nucleic acid molecule of the present invention comprises inverted terminal repeats (ITRs) sequences that are required for genome replication and packaging. In some embodiments, the recombinant nucleic acid molecule of the present invention comprises the AAV2 inverted terminal repeat sequences of SEQ ID NO:12.

SEQ ID NO: 12
> Inverted terminal repeats (ITRs) that are
required for genome replication and packaging.
ctgcgcgctcgctcgctcactgaggccgcccgggcaaagcccgggcgtcg
ggcgacctttggtcgcccggcctcagtgagcgagcgagcgcgcagagagg
gagtggccaactccatcactaggggttcct

In some embodiments, the recombinant nucleic acid molecule of the present invention comprises a nucleic acid sequence selected from the group consisting of SEQ ID NO:13 to SEQ ID NO:24.

SEQ ID NO: 13
> ITR-EF1a-BCKDHA-WT-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagct
agcgctccggtgcccgtcagtgggcagagcgcacatcgcccacagtccccgagaagttggggggagggg
tcggcaattgaaccggtgcctagagaaggtggcgcggggtaaactgggaaagtgatgtcgtgtactggc
tccgcctttttcccgagggtgggggagaaccgtatataagtgcagtagtcgccgtgaacgttctttttc
gcaacgggtttgccgccagaacacaggtaagtgccgtgtgtggttcccgcgggcctggcctctttacgg
gttatggcccttgcgtgccttgaattacttccacgcccctggctgcagtacgtgattcttgatcccgag
cttcgggttggaagtgggtgggagagttcgaggccttgcgcttaaggagccccttcgcctcgtgcttga
gttgaggcctggcctgggcgctggggccgccgcgtgcgaatctggtggcaccttcgcgcctgtctcgct
gctttcgataagtctctagccatttaaaatttttgatgacctgctgcgacgctttttttctggcaagat
agtcttgtaaatgcgggccaagatctgcacactggtatttcggtttttggggccgcgggcggcgacggg
gcccgtgcgtcccagcgcacatgttcggcgaggcggggcctgcgagcgcggccaccgagaatcggacgg
gggtagtctcaagctggccggcctgctctggtgcctggcctcgcgccgccgtgtatcgccccgccctgg
gcggcaaggctggcccggtcggcaccagttgcgtgagcggaaagatggccgcttcccggccctgctgca
gggagctcaaaatggaggacgcggcgctcgggagagcgggcgggtgagtcacccacacaaaggaaaagg
gcctttccgtcctcagccgtcgcttcatgtgactccacggagtaccgggcgccgtccaggcacctcgat
tagttctcgagcttttggagtacgtcgtctttaggttggggggaggggttttatgcgatggagtttccc
cacactgagtgggtggagactgaagttaggccagcttggcacttgatgtaattctccttggaatttgcc
ctttttgagtttggatcttggttcattctcaagcctcagacagtggttcaaagtttttttcttccattt
caggtgtcgtgaagcaattgcgcGGCCGCCATGGCGGTAGCGATCGCTGCAGCGAGGGTCTGGCGGCTA
AACCGTGGTTTGAGCCAGGCTGCCCTCCTGCTGCTGCGGCAGCCTGGGGCTCGGGGACTGGCTAGATCT
CACCCCCCCAGGCAGCAGCAGCAGTTTTCATCTCTGGATGACAAGCCCCAGTTCCCAGGGGCCTCGGCG
GAGTTTATAGATAAGTTGGAATTCATCCAGCCCAACGTCATCTCTGGAATCCCCATCTACCGCGTCATG
GACCGGCAAGGCCAGATCATCAACCCCAGCGAGGACCCCCACCTGCCGAAGGAGAAGGTGCTGAAGCTC
TACAAGAGCATGACACTGCTTAACACCATGGACCGCATCCTCTATGAGTCTCAGCGGCAGGGCCGGATC
TCCTTCTACATGACCAACTATGGTGAGGAGGGCACGCACGTGGGGAGTGCCGCCGCCCTGGACAACACG
GACCTGGTGTTTGGCCAGTACCGGGAGGCAGGTGTGCTGATGTATCGGGACTACCCCCTGGAACTATTC
ATGGCCCAGTGCTATGGCAACATCAGTGACTTGGGCAAGGGGCGCCAGATGCCTGTCCACTACGGCTGC
AAGGAACGCCACTTCGTCACTATCTCCTCTCCACTGGCCACGCAGATCCCTCAGGCGGTGGGGGCGGCG
TACGCAGCCAAGCGGGCCAATGCCAACAGGGTCGTCATCTGTTACTTCGGCGAGGGGGCAGCCAGTGAG
GGGGACGCCCATGCCGGCTTCAACTTCGCTGCCACACTTGAGTGCCCCATCATCTTCTTCTGCCGGAAC
AATGGCTACGCCATCTCCACGCCCACCTCTGAGCAGTATCGCGGCGATGGCATTGCAGCACGAGGCCCC
GGGTATGGCATCATGTCAATCCGCGTGGATGGTAATGATGTGTTTGCCGTATACAACGCCACAAAGGAG
GCCCGACGGCGGGCTGTGGCAGAGAACCAGCCCTTCCTCATCGAGGCCATGACCTACAGGATCGGGCAC
CACAGCACCAGTGACGACAGTTCAGCGTACCGCTCGGTGGATGAGGTCAATTACTGGGATAAACAGGAC
CACCCCATCTCCCGGCTGCGGCACTATCTGCTGAGCCAAGGCTGGTGGGATGAGGAGCAGGAGAAGGCC
TGGAGGAAGCAGTCCCGCAGGAAGGTGATGGAGGCCTTTGAGCAGGCCGAGCGGAAGCCCAAACCCAAC
CCCAACCTACTCTTCTCAGACGTGTATCAGGAGATGCCCGCCCAGCTCCGCAAGCAGCAGGAGTCTCTG
GCCCGCCACCTGCAGACCTACGGGGAGCACTACCCACTGGATCACTTCGATAAGTGAAagcttggatcc
aatcaacctctggattacaaaatttgtgaaagattgactggtattcttaactatgttgctccttttacg
ctatgtggatacgctgctttaatgcctttgtatcatgctattgcttcccgtatggctttcattttctcc
tccttgtataaatcctggttgctgtctctttatgaggagttgtggcccgttgtcaggcaacgtggcgtg
gtgtgcactgtgtttgctgacgcaacccccactggttggggcattgccaccacctgtcagctcctttcc
gggactttcgctttccccctccctattgccacggcggaactcatcgccgcctgccttgcccgctgctgg
acaggggctcggctgttgggcactgacaattccgtggtgttgtcggggaaatcatcgtcctttccttgg
ctgctcgcctgtgttgccacctggattctgcgcgggacgtccttctgctacgtcccttcggccctcaat
ccagcggaccttccttcccgcggcctgctgccggctctgcggcctcttccgcgtcttcgagatctgcct
cgactgtgccttctagttgccagccatctgttgtttgcccctcccccgtgccttccttgaccctggaag
gtgccactcccactgtcctttcctaataaaatgaggaaattgcatcgcattgtctgagtaggtgtcatt
ctattctggggggtggggtggggcaggacagcaagggggaggattgggaagacaatagcaggcatgctg
gggactcgagttaagggcgaattcccgataaggatcttcctagagcatggctacgtagataagtagcat
ggcgggttaatcattaactacaaggaacccctagtgatggagttggccactccctctctgcgcgctcgc
tcgctcactgaggccgggcgaccaaaggtcgcccgacgcccgggctttgcccgggcggcctcagtgagc
gagcgagcgcgcag
SEQ ID NO: 14
> ITR-EF1a-BOKDHA-co1-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagct
agcgctccggtgcccgtcagtgggcagagcgcacatcgcccacagtccccgagaagttggggggagggg
tcggcaattgaaccggtgcctagagaaggtggcgcggggtaaactgggaaagtgatgtcgtgtactggc
tccgcctttttcccgagggtgggggagaaccgtatataagtgcagtagtcgccgtgaacgttctttttc
gcaacgggtttgccgccagaacacaggtaagtgccgtgtgtggttcccgcgggcctggcctctttacgg
gttatggcccttgcgtgccttgaattacttccacgcccctggctgcagtacgtgattcttgatcccgag
cttcgggttggaagtgggtgggagagttcgaggccttgcgcttaaggagccccttcgcctcgtgcttga
gttgaggcctggcctgggcgctggggccgccgcgtgcgaatctggtggcaccttcgcgcctgtctcgct
gctttcgataagtctctagccatttaaaatttttgatgacctgctgcgacgctttttttctggcaagat
agtcttgtaaatgcgggccaagatctgcacactggtatttcggtttttggggccgcgggcggcgacggg
gcccgtgcgtcccagcgcacatgttcggcgaggcggggcctgcgagcgcggccaccgagaatcggacgg
gggtagtctcaagctggccggcctgctctggtgcctggcctcgcgccgccgtgtatcgccccgccctgg
gcggcaaggctggcccggtcggcaccagttgcgtgagcggaaagatggccgcttcccggccctgctgca
gggagctcaaaatggaggacgcggcgctcgggagagcgggcgggtgagtcacccacacaaaggaaaagg
gcctttccgtcctcagccgtcgcttcatgtgactccacggagtaccgggcgccgtccaggcacctcgat
tagttctcgagcttttggagtacgtcgtctttaggttggggggaggggttttatgcgatggagtttccc
cacactgagtgggtggagactgaagttaggccagcttggcacttgatgtaattctccttggaatttgcc
ctttttgagtttggatcttggttcattctcaagcctcagacagtggttcaaagtttttttcttccattt
caggtgtcgtgaagcaattgcgcGGCCGCCATGGCCGTTGCTATCGCTGCCGCGAGAGTATGGCGACTT
AACAGGGGTCTTTCACAAGCTGCTCTTCTTCTTTTGCGACAGCCAGGCGCGCGGGGGCTTGCCCGGAGC
CATCCCCCCCGGCAGCAACAACAGTTCAGTAGCCTTGACGATAAACCGCAATTCCCAGGCGCTTCAGCA
GAGTTCATTGATAAGCTGGAATTCATTCAACCCAACGTAATTTCCGGCATTCCTATTTATCGCGTAATG
GATAGACAGGGGCAAATAATTAACCCGAGCGAGGATCCACATCTTCCCAAGGAAAAAGTTCTTAAATTG
TATAAGTCTATGACCTTGCTTAACACGATGGACCGAATACTCTATGAATCTCAGCGGCAGGGCAGGATT
AGTTTCTATATGACAAATTATGGCGAAGAAGGAACCCACGTCGGGTCCGCAGCGGCCTTGGATAACACC
GACTTGGTCTTTGGACAGTACCGGGAGGCAGGTGTTCTTATGTACCGGGACTATCCCCTTGAGCTGTTC
ATGGCTCAATGTTATGGGAACATTAGTGATCTGGGGAAAGGCCGACAAATGCCCGTGCATTACGGATGT
AAAGAAAGGCATTTTGTAACTATCTCAAGTCCTCTTGCTACTCAAATACCGCAGGCCGTAGGTGCGGCG
TATGCTGCTAAGAGGGCAAACGCCAATAGAGTTGTGATATGCTACTTCGGTGAGGGGGCTGCAAGCGAG
GGAGATGCCCACGCCGGGTTCAACTTTGCAGCGACACTGGAGTGTCCCATTATATTTTTTTGTCGAAAC
AATGGCTATGCGATCTCTACCCCAACATCAGAGCAGTACAGAGGAGATGGGATTGCAGCACGGGGCCCC
GGTTATGGAATCATGTCTATACGCGTGGATGGGAACGACGTCTTTGCCGTATATAACGCTACTAAAGAG
GCCAGAAGACGAGCCGTGGCCGAGAATCAACCCTTCCTTATAGAGGCCATGACTTACAGAATTGGTCAT
CACTCTACGTCCGATGATTCTTCAGCTTACCGCAGCGTGGACGAGGTAAATTACTGGGATAAACAGGAC
CATCCTATTTCACGACTTCGGCATTATCTCCTCAGCCAGGGCTGGTGGGACGAAGAACAAGAAAAGGCA
TGGAGAAAACAATCTAGAAGAAAGGTTATGGAGGCCTTTGAGCAGGCAGAACGCAAACCAAAACCAAAT
CCCAATCTTCTTTTCAGCGACGTGTACCAGGAAATGCCAGCCCAGCTGCGGAAACAGCAAGAAAGCCTG
GCGAGACATCTTCAGACCTACGGGGAACATTACCCACTGGATCACTTTGACAAATGAAagcttggatcc
aatcaacctctggattacaaaatttgtgaaagattgactggtattcttaactatgttgctccttttacg
ctatgtggatacgctgctttaatgcctttgtatcatgctattgcttcccgtatggctttcattttctcc
tccttgtataaatcctggttgctgtctctttatgaggagttgtggcccgttgtcaggcaacgtggcgtg
gtgtgcactgtgtttgctgacgcaacccccactggttggggcattgccaccacctgtcagctcctttcc
gggactttcgctttccccctccctattgccacggcggaactcatcgccgcctgccttgcccgctgctgg
acaggggctcggctgttgggcactgacaattccgtggtgttgtcggggaaatcatcgtcctttccttgg
ctgctcgcctgtgttgccacctggattctgcgcgggacgtccttctgctacgtcccttcggccctcaat
ccagcggaccttccttcccgcggcctgctgccggctctgcggcctcttccgcgtcttcgagatctgcct
cgactgtgccttctagttgccagccatctgttgtttgcccctcccccgtgccttccttgaccctggaag
gtgccactcccactgtcctttcctaataaaatgaggaaattgcatcgcattgtctgagtaggtgtcatt
ctattctggggggtggggtggggcaggacagcaagggggaggattgggaagacaatagcaggcatgctg
gggactcgagttaagggcgaattcccgataaggatcttcctagagcatggctacgtagataagtagcat
ggcgggttaatcattaactacaaggaacccctagtgatggagttggccactccctctctgcgcgctcgc
tcgctcactgaggccgggcgaccaaaggtcgcccgacgcccgggctttgcccgggcggcctcagtgagc
gagcgagcgcgcagc
SEQ ID NO: 15
> ITR-EF1a-BCKDHA-co2-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagct
agcgctccggtgcccgtcagtgggcagagcgcacatcgcccacagtccccgagaagttggggggagggg
tcggcaattgaaccggtgcctagagaaggtggcgcggggtaaactgggaaagtgatgtcgtgtactggc
tccgcctttttcccgagggtgggggagaaccgtatataagtgcagtagtcgccgtgaacgttctttttc
gcaacgggtttgccgccagaacacaggtaagtgccgtgtgtggttcccgcgggcctggcctctttacgg
gttatggcccttgcgtgccttgaattacttccacgcccctggctgcagtacgtgattcttgatcccgag
cttcgggttggaagtgggtgggagagttcgaggccttgcgcttaaggagccccttcgcctcgtgcttga
gttgaggcctggcctgggcgctggggccgccgcgtgcgaatctggtggcaccttcgcgcctgtctcgct
gctttcgataagtctctagccatttaaaatttttgatgacctgctgcgacgctttttttctggcaagat
agtcttgtaaatgcgggccaagatctgcacactggtatttcggtttttggggccgcgggcggcgacggg
gcccgtgcgtcccagcgcacatgttcggcgaggcggggcctgcgagcgcggccaccgagaatcggacgg
gggtagtctcaagctggccggcctgctctggtgcctggcctcgcgccgccgtgtatcgccccgccctgg
gcggcaaggctggcccggtcggcaccagttgcgtgagcggaaagatggccgcttcccggccctgctgca
gggagctcaaaatggaggacgcggcgctcgggagagcgggcgggtgagtcacccacacaaaggaaaagg
gcctttccgtcctcagccgtcgcttcatgtgactccacggagtaccgggcgccgtccaggcacctcgat
tagttctcgagcttttggagtacgtcgtctttaggttggggggaggggttttatgcgatggagtttccc
cacactgagtgggtggagactgaagttaggccagcttggcacttgatgtaattctccttggaatttgcc
ctttttgagtttggatcttggttcattctcaagcctcagacagtggttcaaagtttttttcttccattt
caggtgtcgtgaagcaattgcgcGGCCGCCATGGCTGTTGCTATTGCTGCTGCGAGAGTATGGCGACTT
AACAGGGGTCTTTCACAAGCTGCTCTTCTTCTTTTGAGGCAGCCAGGAGCCAGAGGGCTTGCCAGAAGC
CATCCCCCCAGACAGCAACAACAGTTCAGTAGCCTTGATGATAAACCCCAATTCCCAGGAGCTTCAGCA
GAGTTCATTGATAAGCTGGAATTCATTCAACCCAATGTAATTTCTGGCATTCCTATTTATAGAGTAATG
GATAGACAGGGGCAAATAATTAACCCCTCCGAGGATCCACATCTTCCCAAGGAAAAAGTTCTTAAATTG
TATAAGTCTATGACCTTGCTTAACACCATGGACAGGATACTCTATGAATCTCAGAGACAGGGCAGGATT
AGTTTCTATATGACAAATTATGGAGAAGAAGGAACCCACGTGGGGAGCGCAGCCGCCTTGGATAACACC
GACTTGGTCTTTGGACAGTACAGGGAGGCAGGTGTTCTTATGTACAGGGACTATCCCCTTGAGCTGTTC
ATGGCTCAATGTTATGGGAACATTAGTGATCTGGGGAAAGGCCGACAAATGCCCGTGCATTACGGATGT
AAAGAAAGGCATTTTGTAACTATCTCAAGTCCTCTTGCTACTCAAATACCCCAGGCTGTAGGTGCCGCC
TATGCTGCTAAGAGGGCAAACGCCAATAGAGTTGTGATATGCTACTTCGGTGAGGGGGCTGCAAGCGAG
GGAGATGCCCACGCTGGGTTCAACTTTGCAGCCACACTGGAGTGTCCCATTATATTTTTTTGTAGGAAC
AATGGCTATGCCATCTCTACCCCAACATCAGAGCAGTACAGAGGAGATGGGATTGCAGCAAGAGGCCCC
GGTTATGGAATCATGTCTATAAGGGTGGATGGGAACGACGTCTTTGCCGTGTATAACGCTACTAAAGAG
GCCAGAAGAAGGGCTGTGGCTGAGAATCAACCCTTCCTTATAGAGGCCATGACTTACAGAATTGGTCAT
CACTCTACCTCCGATGATTCTTCAGCTTACAGATCCGTGGATGAGGTAAATTACTGGGATAAACAGGAC
CATCCTATTTCAAGACTTAGGCATTATCTCCTCAGCCAGGGCTGGTGGGACGAAGAACAAGAAAAGGCA
TGGAGAAAACAATCTAGAAGAAAGGTTATGGAGGCCTTTGAGCAGGCAGAAAGGAAACCAAAACCAAAT
CCCAATCTTCTTTTCTCCGACGTGTACCAGGAAATGCCAGCCCAGCTGAGGAAACAGCAAGAAAGCCTG
GCCAGACATCTTCAGACCTACGGGGAACATTACCCACTGGATCACTTTGACAAATGAAagcttggatcc
aatcaacctctggattacaaaatttgtgaaagattgactggtattcttaactatgttgctccttttacg
ctatgtggatacgctgctttaatgcctttgtatcatgctattgcttcccgtatggctttcattttctcc
tccttgtataaatcctggttgctgtctctttatgaggagttgtggcccgttgtcaggcaacgtggcgtg
gtgtgcactgtgtttgctgacgcaacccccactggttggggcattgccaccacctgtcagctcctttcc
gggactttcgctttccccctccctattgccacggcggaactcatcgccgcctgccttgcccgctgctgg
acaggggctcggctgttgggcactgacaattccgtggtgttgtcggggaaatcatcgtcctttccttgg
ctgctcgcctgtgttgccacctggattctgcgcgggacgtccttctgctacgtcccttcggccctcaat
ccagcggaccttccttcccgcggcctgctgccggctctgcggcctcttccgcgtcttcgagatctgcct
cgactgtgccttctagttgccagccatctgttgtttgcccctcccccgtgccttccttgaccctggaag
gtgccactcccactgtcctttcctaataaaatgaggaaattgcatcgcattgtctgagtaggtgtcatt
ctattctggggggtggggtggggcaggacagcaagggggaggattgggaagacaatagcaggcatgctg
gggactcgagttaagggcgaattcccgataaggatcttcctagagcatggctacgtagataagtagcat
ggcgggttaatcattaactacaaggaacccctagtgatggagttggccactccctctctgcgcgctcgc
tcgctcactgaggccgggcgaccaaaggtcgcccgacgcccgggctttgcccgggcggcctcagtgagc
gagcgagcgcgcag
SEQ ID NO: 16
> ITR-EF1a-BCKDHB-WT-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagct
agcgctccggtgcccgtcagtgggcagagcgcacatcgcccacagtccccgagaagttggggggagggg
tcggcaattgaaccggtgcctagagaaggtggcgcggggtaaactgggaaagtgatgtcgtgtactggc
tccgcctttttcccgagggtgggggagaaccgtatataagtgcagtagtcgccgtgaacgttctttttc
gcaacgggtttgccgccagaacacaggtaagtgccgtgtgtggttcccgcgggcctggcctctttacgg
gttatggcccttgcgtgccttgaattacttccacgcccctggctgcagtacgtgattcttgatcccgag
cttcgggttggaagtgggtgggagagttcgaggccttgcgcttaaggagccccttcgcctcgtgcttga
gttgaggcctggcctgggcgctggggccgccgcgtgcgaatctggtggcaccttcgcgcctgtctcgct
gctttcgataagtctctagccatttaaaatttttgatgacctgctgcgacgctttttttctggcaagat
agtcttgtaaatgcgggccaagatctgcacactggtatttcggtttttggggccgcgggcggcgacggg
gcccgtgcgtcccagcgcacatgttcggcgaggcggggcctgcgagcgcggccaccgagaatcggacgg
gggtagtctcaagctggccggcctgctctggtgcctggcctcgcgccgccgtgtatcgccccgccctgg
gcggcaaggctggcccggtcggcaccagttgcgtgagcggaaagatggccgcttcccggccctgctgca
gggagctcaaaatggaggacgcggcgctcgggagagcgggcgggtgagtcacccacacaaaggaaaagg
gcctttccgtcctcagccgtcgcttcatgtgactccacggagtaccgggcgccgtccaggcacctcgat
tagttctcgagcttttggagtacgtcgtctttaggttggggggaggggttttatgcgatggagtttccc
cacactgagtgggtggagactgaagttaggccagcttggcacttgatgtaattctccttggaatttgcc
ctttttgagtttggatcttggttcattctcaagcctcagacagtggttcaaagtttttttcttccattt
caggtgtcgtgaagcaattgcgcGGCCGCCATGGCGGTTGTAGCGGCGGCTGCCGGCTGGCTACTCAGG
CTCAGGGCGGCAGGGGCTGAGGGGCACTGGCGTCGGCTTCCTGGCGCGGGGCTGGCGCGGGGCTTTTTG
CACCCCGCCGCGACTGTCGAGGATGCGGCCCAGAGGCGGCAGGTGGCTCATTTTACTTTCCAGCCAGAT
CCGGAGCCCCGGGAGTACGGGCAAACTCAGAAAATGAATCTTTTCCAGTCTGTAACAAGTGCCTTGGAT
AACTCATTGGCCAAAGATCCTACTGCAGTAATATTTGGTGAAGATGTTGCCTTTGGTGGAGTCTTTAGA
TGCACTGTTGGCTTGCGAGACAAATATGGAAAAGATAGAGTTTTTAATACCCCATTGTGTGAACAAGGA
ATTGTTGGATTTGGAATCGGAATTGCGGTCACTGGAGCTACTGCCATTGCGGAAATTCAGTTTGCAGAT
TATATTTTCCCTGCATTTGATCAGATTGTTAATGAAGCTGCCAAGTATCGCTATCGCTCTGGGGATCTT
TTTAACTGTGGAAGCCTCACTATCCGGTCCCCTTGGGGCTGTGTTGGTCATGGGGCTCTCTATCATTCT
CAGAGTCCTGAAGCATTTTTTGCCCATTGCCCAGGAATCAAGGTGGTTATACCCAGAAGCCCTTTCCAG
GCCAAAGGACTTCTTTTGTCATGCATAGAGGATAAAAATCCTTGTATATTTTTTGAACCTAAAATACTT
TACAGGGCAGCAGCGGAAGAAGTCCCTATAGAACCATACAACATCCCACTGTCCCAGGCCGAAGTCATA
CAGGAAGGGAGTGATGTTACTCTAGTTGCCTGGGGCACTCAGGTTCATGTGATCCGAGAGGTAGCTTCC
ATGGCAAAAGAAAAGCTTGGAGTGTCTTGTGAAGTCATTGATCTGAGGACTATAATACCTTGGGATGTG
GACACAATTTGTAAGTCTGTGATCAAAACAGGGCGACTGCTAATCAGTCACGAGGCTCCCTTGACAGGC
GGCTTTGCATCGGAAATCAGCTCTACAGTTCAGGAGGAATGTTTCTTGAACCTAGAGGCTCCTATATCA
AGAGTATGTGGTTATGACACACCATTTCCTCACATTTTTGAACCATTCTACATCCCAGACAAATGGAAG
TGTTATGATGCCCTTCGAAAAATGATCAACTATTGAGgatccaatcaacctctggattacaaaatttgt
gaaagattgactggtattcttaactatgttgctccttttacgctatgtggatacgctgctttaatgcct
ttgtatcatgctattgcttcccgtatggctttcattttctcctccttgtataaatcctggttgctgtct
ctttatgaggagttgtggcccgttgtcaggcaacgtggcgtggtgtgcactgtgtttgctgacgcaacc
cccactggttggggcattgccaccacctgtcagctcctttccgggactttcgctttccccctccctatt
gccacggcggaactcatcgccgcctgccttgcccgctgctggacaggggctcggctgttgggcactgac
aattccgtggtgttgtcggggaaatcatcgtcctttccttggctgctcgcctgtgttgccacctggatt
ctgcgcgggacgtccttctgctacgtcccttcggccctcaatccagcggaccttccttcccgcggcctg
ctgccggctctgcggcctcttccgcgtcttcgagatctgcctcgactgtgccttctagttgccagccat
ctgttgtttgcccctcccccgtgccttccttgaccctggaaggtgccactcccactgtcctttcctaat
aaaatgaggaaattgcatcgcattgtctgagtaggtgtcattctattctggggggtggggtggggcagg
acagcaagggggaggattgggaagacaatagcaggcatgctggggactcgagttaagggcgaattcccg
ataaggatcttcctagagcatggctacgtagataagtagcatggcgggttaatcattaactacaaggaa
cccctagtgatggagttggccactccctctctgcgcgctcgctcgctcactgaggccgggcgaccaaag
gtcgcccgacgcccgggctttgcccgggcggcctcagtgagcgagcgagcgcgcag
SEQ ID NO: 17
> ITR-EF1a-BCKDHB-co1-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagct
agcgctccggtgcccgtcagtgggcagagcgcacatcgcccacagtccccgagaagttggggggagggg
tcggcaattgaaccggtgcctagagaaggtggcgcggggtaaactgggaaagtgatgtcgtgtactggc
tccgcctttttcccgagggtgggggagaaccgtatataagtgcagtagtcgccgtgaacgttctttttc
gcaacgggtttgccgccagaacacaggtaagtgccgtgtgtggttcccgcgggcctggcctctttacgg
gttatggcccttgcgtgccttgaattacttccacgcccctggctgcagtacgtgattcttgatcccgag
cttcgggttggaagtgggtgggagagttcgaggccttgcgcttaaggagccccttcgcctcgtgcttga
gttgaggcctggcctgggcgctggggccgccgcgtgcgaatctggtggcaccttcgcgcctgtctcgct
gctttcgataagtctctagccatttaaaatttttgatgacctgctgcgacgctttttttctggcaagat
agtcttgtaaatgcgggccaagatctgcacactggtatttcggtttttggggccgcgggcggcgacggg
gcccgtgcgtcccagcgcacatgttcggcgaggcggggcctgcgagcgcggccaccgagaatcggacgg
gggtagtctcaagctggccggcctgctctggtgcctggcctcgcgccgccgtgtatcgccccgccctgg
gcggcaaggctggcccggtcggcaccagttgcgtgagcggaaagatggccgcttcccggccctgctgca
gggagctcaaaatggaggacgcggcgctcgggagagcgggcgggtgagtcacccacacaaaggaaaagg
gcctttccgtcctcagccgtcgcttcatgtgactccacggagtaccgggcgccgtccaggcacctcgat
tagttctcgagcttttggagtacgtcgtctttaggttggggggaggggttttatgcgatggagtttccc
cacactgagtgggtggagactgaagttaggccagcttggcacttgatgtaattctccttggaatttgcc
ctttttgagtttggatcttggttcattctcaagcctcagacagtggttcaaagtttttttcttccattt
caggtgtcgtgaagcaattgcgcGGCCGCCATGGCAGTTGTGGCAGCCGCAGCGGGCTGGTTGTTGCGA
CTCAGAGCAGCCGGTGCAGAAGGCCATTGGAGACGGTTGCCGGGTGCGGGACTGGCGCGCGGCTTTCTC
CACCCCGCAGCGACTGTAGAAGACGCAGCCCAAAGACGACAGGTCGCTCACTTCACATTCCAGCCTGAT
CCCGAGCCACGAGAATACGGGCAAACGCAAAAAATGAATCTCTTTCAGTCCGTAACATCTGCTTTGGAT
AATAGTCTTGCAAAAGATCCAACAGCTGTAATTTTCGGGGAAGATGTAGCGTTTGGCGGTGTCTTCCGA
TGTACCGTCGGGCTGAGGGATAAGTACGGGAAAGATAGAGTATTTAATACCCCCCTGTGCGAGCAGGGT
ATAGTCGGATTTGGGATTGGAATAGCCGTAACGGGAGCAACAGCGATTGCCGAAATACAATTTGCCGAC
TATATCTTCCCGGCGTTTGACCAAATTGTTAACGAGGCTGCGAAATATCGGTATCGCTCCGGCGACTTG
TTTAATTGCGGTAGCCTCACAATTAGAAGTCCTTGGGGGTGCGTTGGACACGGTGCGCTCTATCACAGT
CAATCTCCAGAAGCTTTTTTCGCACATTGTCCAGGCATCAAAGTAGTGATTCCCCGAAGCCCATTTCAG
GCGAAAGGTCTCTTGCTCTCCTGTATAGAAGATAAAAACCCATGTATCTTTTTTGAGCCTAAAATCCTG
TACCGCGCCGCAGCTGAGGAAGTCCCTATAGAGCCATACAACATCCCACTCTCACAGGCAGAAGTTATA
CAAGAAGGGAGTGACGTGACACTCGTAGCATGGGGGACGCAGGTTCATGTGATCAGAGAGGTAGCCAGT
ATGGCAAAAGAGAAATTGGGAGTTTCTTGTGAAGTTATCGATCTCCGAACAATAATCCCTTGGGATGTA
GATACCATTTGTAAGTCTGTTATCAAAACTGGTAGGCTCCTCATATCTCATGAGGCCCCGTTGACGGGT
GGGTTCGCGTCCGAAATTTCATCAACTGTTCAAGAGGAGTGCTTTCTCAACCTGGAAGCGCCGATCTCT
AGAGTCTGCGGATATGATACCCCCTTCCCACACATATTTGAGCCTTTTTATATCCCGGACAAATGGAAG
TGTTACGACGCCCTTCGAAAAATGATAAATTATTGAGgatccaatcaacctctggattacaaaatttgt
gaaagattgactggtattcttaactatgttgctccttttacgctatgtggatacgctgctttaatgcct
ttgtatcatgctattgcttcccgtatggctttcattttctcctccttgtataaatcctggttgctgtct
ctttatgaggagttgtggcccgttgtcaggcaacgtggcgtggtgtgcactgtgtttgctgacgcaacc
cccactggttggggcattgccaccacctgtcagctcctttccgggactttcgctttccccctccctatt
gccacggcggaactcatcgccgcctgccttgcccgctgctggacaggggctcggctgttgggcactgac
aattccgtggtgttgtcggggaaatcatcgtcctttccttggctgctcgcctgtgttgccacctggatt
ctgcgcgggacgtccttctgctacgtcccttcggccctcaatccagcggaccttccttcccgcggcctg
ctgccggctctgcggcctcttccgcgtcttcgagatctgcctcgactgtgccttctagttgccagccat
ctgttgtttgcccctcccccgtgccttccttgaccctggaaggtgccactcccactgtcctttcctaat
aaaatgaggaaattgcatcgcattgtctgagtaggtgtcattctattctggggggtggggtggggcagg
acagcaagggggaggattgggaagacaatagcaggcatgctggggactcgagttaagggcgaattcccg
ataaggatcttcctagagcatggctacgtagataagtagcatggcgggttaatcattaactacaaggaa
cccctagtgatggagttggccactccctctctgcgcgctcgctcgctcactgaggccgggcgaccaaag
gtcgcccgacgcccgggctttgcccgggcggcctcagtgagcgagcgagcgcgcag
SEQ ID NO: 18
> ITR-EF1a-BCKDHB-co2-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagct
agcgctccggtgcccgtcagtgggcagagcgcacatcgcccacagtccccgagaagttggggggagggg
tcggcaattgaaccggtgcctagagaaggtggcgcggggtaaactgggaaagtgatgtcgtgtactggc
tccgcctttttcccgagggtgggggagaaccgtatataagtgcagtagtcgccgtgaacgttctttttc
gcaacgggtttgccgccagaacacaggtaagtgccgtgtgtggttcccgcgggcctggcctctttacgg
gttatggcccttgcgtgccttgaattacttccacgcccctggctgcagtacgtgattcttgatcccgag
cttcgggttggaagtgggtgggagagttcgaggccttgcgcttaaggagccccttcgcctcgtgcttga
gttgaggcctggcctgggcgctggggccgccgcgtgcgaatctggtggcaccttcgcgcctgtctcgct
gctttcgataagtctctagccatttaaaatttttgatgacctgctgcgacgctttttttctggcaagat
agtcttgtaaatgcgggccaagatctgcacactggtatttcggtttttggggccgcgggcggcgacggg
gcccgtgcgtcccagcgcacatgttcggcgaggcggggcctgcgagcgcggccaccgagaatcggacgg
gggtagtctcaagctggccggcctgctctggtgcctggcctcgcgccgccgtgtatcgccccgccctgg
gcggcaaggctggcccggtcggcaccagttgcgtgagcggaaagatggccgcttcccggccctgctgca
gggagctcaaaatggaggacgcggcgctcgggagagcgggcgggtgagtcacccacacaaaggaaaagg
gcctttccgtcctcagccgtcgcttcatgtgactccacggagtaccgggcgccgtccaggcacctcgat
tagttctcgagcttttggagtacgtcgtctttaggttggggggaggggttttatgcgatggagtttccc
cacactgagtgggtggagactgaagttaggccagcttggcacttgatgtaattctccttggaatttgcc
ctttttgagtttggatcttggttcattctcaagcctcagacagtggttcaaagtttttttcttccattt
caggtgtcgtgaagcaattgcgcGGCCGCCATGGCAGTTGTGGCAGCTGCAGCAGGCTGGTTGTTGCGC
CTCAGAGCAGCTGGTGCAGAAGGCCATTGGAGAAGGTTGCCTGGTGCCGGACTGGCCCGCGGCTTTCTC
CACCCCGCAGCCACTGTAGAAGATGCAGCCCAAAGAAGACAGGTCGCTCACTTCACATTCCAGCCTGAT
CCCGAGCCAAGAGAATATGGGCAAACCCAAAAAATGAATCTCTTTCAGTCCGTAACATCTGCTTTGGAT
AATAGTCTTGCAAAAGATCCAACAGCTGTAATTTTCGGGGAAGATGTAGCATTTGGAGGTGTCTTCAGG
TGTACAGTCGGGCTGAGGGATAAGTACGGGAAAGATAGAGTATTTAATACCCCCCTGTGTGAGCAGGGT
ATAGTGGGATTTGGGATTGGAATAGCTGTAACGGGAGCAACAGCAATTGCTGAAATACAATTTGCTGAC
TATATCTTCCCGGCATTTGACCAAATTGTTAACGAGGCTGCAAAATATAGGTATAGGTCCGGAGACTTG
TTTAATTGTGGTAGCCTCACAATTAGAAGTCCTTGGGGGTGTGTTGGACATGGTGCACTCTATCACAGT
CAATCTCCAGAAGCTTTTTTCGCACATTGTCCAGGCATCAAAGTAGTGATTCCCAGGAGCCCATTTCAG
GCAAAAGGTCTCTTGCTCTCCTGTATAGAAGATAAAAACCCATGTATCTTTTTTGAGCCTAAAATCCTG
TACAGAGCTGCAGCTGAGGAAGTCCCTATAGAGCCATACAACATCCCACTCTCACAGGCAGAAGTTATA
CAAGAAGGGAGTGATGTGACACTGGTAGCATGGGGGACCCAGGTTCATGTGATCAGAGAGGTAGCCAGT
ATGGCAAAAGAGAAATTGGGAGTTTCTTGTGAAGTTATCGATCTCCGAACAATAATCCCTTGGGATGTA
GATACCATTTGTAAGTCTGTTATCAAAACTGGTAGGCTCCTCATATCTCATGAGGCCCCGTTGACCGGT
GGGTTCGCATCCGAAATTTCATCAACTGTTCAAGAGGAGTGCTTTCTCAACCTGGAAGCACCAATCTCT
AGAGTCTGTGGATATGATACCCCCTTCCCACACATATTTGAGCCTTTTTATATCCCAGACAAATGGAAG
TGTTACGATGCCCTTAGAAAAATGATAAATTATTGAGgatccaatcaacctctggattacaaaatttgt
gaaagattgactggtattcttaactatgttgctccttttacgctatgtggatacgctgctttaatgcct
ttgtatcatgctattgcttcccgtatggctttcattttctcctccttgtataaatcctggttgctgtct
ctttatgaggagttgtggcccgttgtcaggcaacgtggcgtggtgtgcactgtgtttgctgacgcaacc
cccactggttggggcattgccaccacctgtcagctcctttccgggactttcgctttccccctccctatt
gccacggcggaactcatcgccgcctgccttgcccgctgctggacaggggctcggctgttgggcactgac
aattccgtggtgttgtcggggaaatcatcgtcctttccttggctgctcgcctgtgttgccacctggatt
ctgcgcgggacgtccttctgctacgtcccttcggccctcaatccagcggaccttccttcccgcggcctg
ctgccggctctgcggcctcttccgcgtcttcgagatctgcctcgactgtgccttctagttgccagccat
ctgttgtttgcccctcccccgtgccttccttgaccctggaaggtgccactcccactgtcctttcctaat
aaaatgaggaaattgcatcgcattgtctgagtaggtgtcattctattctggggggtggggtggggcagg
acagcaagggggaggattgggaagacaatagcaggcatgctggggactcgagttaagggcgaattcccg
ataaggatcttcctagagcatggctacgtagataagtagcatggcgggttaatcattaactacaaggaa
cccctagtgatggagttggccactccctctctgcgcgctcgctcgctcactgaggccgggcgaccaaag
gtcgcccgacgcccgggctttgcccgggcggcctcagtgagcgagcgagcgcgcag
SEQ ID NO: 19
> ITR-hAAT-BCKDHA-WT-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagCT
AGCAGGCTCAGAGGCACACAGGAGTTTCTGGGCTCACCCTGCCCCCTTCCAACCCCTCAGTTCCCATCC
TCCAGCAGCTGTTTGTGTGCTGCCTCTGAAGTCCACACTGAACAAACTTCAGCCTACTCATGTCCCTAA
AATGGGCAAACATTGCAAGCAGCAAACAGCAAACACACAGCCCTCCCTGCCTGCTGACCTTGGAGCTGG
GGCAGAGGTCAGAGACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGGATCTTGCTACCAGTGGAACA
GCCACTAAGGATTCTGCAGTGAGAGCAGAGGGCCAGCTAAGTGGTACTCTCCCAGAGACTGTCTGACTC
ACGCCACCCCCTCCACCTTGGACACAGGACGCTGTGGTTTCTGAGCCAGGTACAATGACTCCTTTCGGT
AAGTGCAGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCAGCAG
CCTCCCCCGTTGCCCCTCTGGATCCACTGCTTAAATACGGACGAGGACAGGGCCCTGTCTCCTCAGCTT
CAGGCACCACCACTGACCTGGGACAGTGAATGTAAGTATCAAGGTTACAAGACAGGTTTAAGGAGACCA
ATAGAAACTGGGCTTGTCGAGACAGAGAAGACTCTTGCGTTTCTGATAGGCACCTATTGGTCTTACTGA
CATCCACTTTGCCTTTCTCTCCACAGcaattgcGCGGCCGCCATGGCGGTAGCGATCGCTGCAGCGAGG
GTCTGGCGGCTAAACCGTGGTTTGAGCCAGGCTGCCCTCCTGCTGCTGCGGCAGCCTGGGGCTCGGGGA
CTGGCTAGATCTCACCCCCCCAGGCAGCAGCAGCAGTTTTCATCTCTGGATGACAAGCCCCAGTTCCCA
GGGGCCTCGGCGGAGTTTATAGATAAGTTGGAATTCATCCAGCCCAACGTCATCTCTGGAATCCCCATC
TACCGCGTCATGGACCGGCAAGGCCAGATCATCAACCCCAGCGAGGACCCCCACCTGCCGAAGGAGAAG
GTGCTGAAGCTCTACAAGAGCATGACACTGCTTAACACCATGGACCGCATCCTCTATGAGTCTCAGCGG
CAGGGCCGGATCTCCTTCTACATGACCAACTATGGTGAGGAGGGCACGCACGTGGGGAGTGCCGCCGCC
CTGGACAACACGGACCTGGTGTTTGGCCAGTACCGGGAGGCAGGTGTGCTGATGTATCGGGACTACCCC
CTGGAACTATTCATGGCCCAGTGCTATGGCAACATCAGTGACTTGGGCAAGGGGCGCCAGATGCCTGTC
CACTACGGCTGCAAGGAACGCCACTTCGTCACTATCTCCTCTCCACTGGCCACGCAGATCCCTCAGGCG
GTGGGGGCGGCGTACGCAGCCAAGCGGGCCAATGCCAACAGGGTCGTCATCTGTTACTTCGGCGAGGGG
GCAGCCAGTGAGGGGGACGCCCATGCCGGCTTCAACTTCGCTGCCACACTTGAGTGCCCCATCATCTTC
TTCTGCCGGAACAATGGCTACGCCATCTCCACGCCCACCTCTGAGCAGTATCGCGGCGATGGCATTGCA
GCACGAGGCCCCGGGTATGGCATCATGTCAATCCGCGTGGATGGTAATGATGTGTTTGCCGTATACAAC
GCCACAAAGGAGGCCCGACGGCGGGCTGTGGCAGAGAACCAGCCCTTCCTCATCGAGGCCATGACCTAC
AGGATCGGGCACCACAGCACCAGTGACGACAGTTCAGCGTACCGCTCGGTGGATGAGGTCAATTACTGG
GATAAACAGGACCACCCCATCTCCCGGCTGCGGCACTATCTGCTGAGCCAAGGCTGGTGGGATGAGGAG
CAGGAGAAGGCCTGGAGGAAGCAGTCCCGCAGGAAGGTGATGGAGGCCTTTGAGCAGGCCGAGCGGAAG
CCCAAACCCAACCCCAACCTACTCTTCTCAGACGTGTATCAGGAGATGCCCGCCCAGCTCCGCAAGCAG
CAGGAGTCTCTGGCCCGCCACCTGCAGACCTACGGGGAGCACTACCCACTGGATCACTTCGATAAGTGA
Aagcttggatccaatcaacctctggattacaaaatttgtgaaagattgactggtattcttaactatgtt
gctccttttacgctatgtggatacgctgctttaatgcctttgtatcatgctattgcttcccgtatggct
ttcattttctcctccttgtataaatcctggttgctgtctctttatgaggagttgtggcccgttgtcagg
caacgtggcgtggtgtgcactgtgtttgctgacgcaacccccactggttggggcattgccaccacctgt
cagctcctttccgggactttcgctttccccctccctattgccacggcggaactcatcgccgcctgcctt
gcccgctgctggacaggggctcggctgttgggcactgacaattccgtggtgttgtcggggaaatcatcg
tcctttccttggctgctcgcctgtgttgccacctggattctgcgcgggacgtccttctgctacgtccct
tcggccctcaatccagcggaccttccttcccgcggcctgctgccggctctgcggcctcttccgcgtctt
cgagatctgcctcgactgtgccttctagttgccagccatctgttgtttgcccctcccccgtgccttcct
tgaccctggaaggtgccactcccactgtcctttcctaataaaatgaggaaattgcatcgcattgtctga
gtaggtgtcattctattctggggggtggggtggggcaggacagcaagggggaggattgggaagacaata
gcaggcatgctggggactcgagttaagggcgaattcccgataaggatcttcctagagcatggctacgta
gataagtagcatggcgggttaatcattaactacaaggaacccctagtgatggagttggccactccctct
ctgcgcgctcgctcgctcactgaggccgggcgaccaaaggtcgcccgacgcccgggctttgcccgggcg
gcctcagtgagcgagcgagcgcgcag
SEQ ID NO: 20
> ITR-hAAT-BCKDHA-co1-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagCT
AGCAGGCTCAGAGGCACACAGGAGTTTCTGGGCTCACCCTGCCCCCTTCCAACCCCTCAGTTCCCATCC
TCCAGCAGCTGTTTGTGTGCTGCCTCTGAAGTCCACACTGAACAAACTTCAGCCTACTCATGTCCCTAA
AATGGGCAAACATTGCAAGCAGCAAACAGCAAACACACAGCCCTCCCTGCCTGCTGACCTTGGAGCTGG
GGCAGAGGTCAGAGACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGGATCTTGCTACCAGTGGAACA
GCCACTAAGGATTCTGCAGTGAGAGCAGAGGGCCAGCTAAGTGGTACTCTCCCAGAGACTGTCTGACTC
ACGCCACCCCCTCCACCTTGGACACAGGACGCTGTGGTTTCTGAGCCAGGTACAATGACTCCTTTCGGT
AAGTGCAGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCAGCAG
CCTCCCCCGTTGCCCCTCTGGATCCACTGCTTAAATACGGACGAGGACAGGGCCCTGTCTCCTCAGCTT
CAGGCACCACCACTGACCTGGGACAGTGAATGTAAGTATCAAGGTTACAAGACAGGTTTAAGGAGACCA
ATAGAAACTGGGCTTGTCGAGACAGAGAAGACTCTTGCGTTTCTGATAGGCACCTATTGGTCTTACTGA
CATCCACTTTGCCTTTCTCTCCACAGcaattgcGCGGCCGCCATGGCCGTTGCTATCGCTGCCGCGAGA
GTATGGCGACTTAACAGGGGTCTTTCACAAGCTGCTCTTCTTCTTTTGCGACAGCCAGGCGCGCGGGGG
CTTGCCCGGAGCCATCCCCCCCGGCAGCAACAACAGTTCAGTAGCCTTGACGATAAACCGCAATTCCCA
GGCGCTTCAGCAGAGTTCATTGATAAGCTGGAATTCATTCAACCCAACGTAATTTCCGGCATTCCTATT
TATCGCGTAATGGATAGACAGGGGCAAATAATTAACCCGAGCGAGGATCCACATCTTCCCAAGGAAAAA
GTTCTTAAATTGTATAAGTCTATGACCTTGCTTAACACGATGGACCGAATACTCTATGAATCTCAGCGG
CAGGGCAGGATTAGTTTCTATATGACAAATTATGGCGAAGAAGGAACCCACGTCGGGTCCGCAGCGGCC
TTGGATAACACCGACTTGGTCTTTGGACAGTACCGGGAGGCAGGTGTTCTTATGTACCGGGACTATCCC
CTTGAGCTGTTCATGGCTCAATGTTATGGGAACATTAGTGATCTGGGGAAAGGCCGACAAATGCCCGTG
CATTACGGATGTAAAGAAAGGCATTTTGTAACTATCTCAAGTCCTCTTGCTACTCAAATACCGCAGGCC
GTAGGTGCGGCGTATGCTGCTAAGAGGGCAAACGCCAATAGAGTTGTGATATGCTACTTCGGTGAGGGG
GCTGCAAGCGAGGGAGATGCCCACGCCGGGTTCAACTTTGCAGCGACACTGGAGTGTCCCATTATATTT
TTTTGTCGAAACAATGGCTATGCGATCTCTACCCCAACATCAGAGCAGTACAGAGGAGATGGGATTGCA
GCACGGGGCCCCGGTTATGGAATCATGTCTATACGCGTGGATGGGAACGACGTCTTTGCCGTATATAAC
GCTACTAAAGAGGCCAGAAGACGAGCCGTGGCCGAGAATCAACCCTTCCTTATAGAGGCCATGACTTAC
AGAATTGGTCATCACTCTACGTCCGATGATTCTTCAGCTTACCGCAGCGTGGACGAGGTAAATTACTGG
GATAAACAGGACCATCCTATTTCACGACTTCGGCATTATCTCCTCAGCCAGGGCTGGTGGGACGAAGAA
CAAGAAAAGGCATGGAGAAAACAATCTAGAAGAAAGGTTATGGAGGCCTTTGAGCAGGCAGAACGCAAA
CCAAAACCAAATCCCAATCTTCTTTTCAGCGACGTGTACCAGGAAATGCCAGCCCAGCTGCGGAAACAG
CAAGAAAGCCTGGCGAGACATCTTCAGACCTACGGGGAACATTACCCACTGGATCACTTTGACAAATGA
Aagcttggatccaatcaacctctggattacaaaatttgtgaaagattgactggtattcttaactatgtt
gctccttttacgctatgtggatacgctgctttaatgcctttgtatcatgctattgcttcccgtatggct
ttcattttctcctccttgtataaatcctggttgctgtctctttatgaggagttgtggcccgttgtcagg
caacgtggcgtggtgtgcactgtgtttgctgacgcaacccccactggttggggcattgccaccacctgt
cagctcctttccgggactttcgctttccccctccctattgccacggcggaactcatcgccgcctgcctt
gcccgctgctggacaggggctcggctgttgggcactgacaattccgtggtgttgtcggggaaatcatcg
tcctttccttggctgctcgcctgtgttgccacctggattctgcgcgggacgtccttctgctacgtccct
tcggccctcaatccagcggaccttccttcccgcggcctgctgccggctctgcggcctcttccgcgtctt
cgagatctgcctcgactgtgccttctagttgccagccatctgttgtttgcccctcccccgtgccttcct
tgaccctggaaggtgccactcccactgtcctttcctaataaaatgaggaaattgcatcgcattgtctga
gtaggtgtcattctattctggggggtggggtggggcaggacagcaagggggaggattgggaagacaata
gcaggcatgctggggactcgagttaagggcgaattcccgataaggatcttcctagagcatggctacgta
gataagtagcatggcgggttaatcattaactacaaggaacccctagtgatggagttggccactccctct
ctgcgcgctcgctcgctcactgaggccgggcgaccaaaggtcgcccgacgcccgggctttgcccgggcg
gcctcagtgagcgagcgagcgcgcag
SEQ ID NO: 21
> ITR-hAAT-BCKDHA-co2-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagCT
AGCAGGCTCAGAGGCACACAGGAGTTTCTGGGCTCACCCTGCCCCCTTCCAACCCCTCAGTTCCCATCC
TCCAGCAGCTGTTTGTGTGCTGCCTCTGAAGTCCACACTGAACAAACTTCAGCCTACTCATGTCCCTAA
AATGGGCAAACATTGCAAGCAGCAAACAGCAAACACACAGCCCTCCCTGCCTGCTGACCTTGGAGCTGG
GGCAGAGGTCAGAGACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGGATCTTGCTACCAGTGGAACA
GCCACTAAGGATTCTGCAGTGAGAGCAGAGGGCCAGCTAAGTGGTACTCTCCCAGAGACTGTCTGACTC
ACGCCACCCCCTCCACCTTGGACACAGGACGCTGTGGTTTCTGAGCCAGGTACAATGACTCCTTTCGGT
AAGTGCAGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCAGCAG
CCTCCCCCGTTGCCCCTCTGGATCCACTGCTTAAATACGGACGAGGACAGGGCCCTGTCTCCTCAGCTT
CAGGCACCACCACTGACCTGGGACAGTGAATGTAAGTATCAAGGTTACAAGACAGGTTTAAGGAGACCA
ATAGAAACTGGGCTTGTCGAGACAGAGAAGACTCTTGCGTTTCTGATAGGCACCTATTGGTCTTACTGA
CATCCACTTTGCCTTTCTCTCCACAGcaattgcGCGGCCGCCATGGCTGTTGCTATTGCTGCTGCGAGA
GTATGGCGACTTAACAGGGGTCTTTCACAAGCTGCTCTTCTTCTTTTGAGGCAGCCAGGAGCCAGAGGG
CTTGCCAGAAGCCATCCCCCCAGACAGCAACAACAGTTCAGTAGCCTTGATGATAAACCCCAATTCCCA
GGAGCTTCAGCAGAGTTCATTGATAAGCTGGAATTCATTCAACCCAATGTAATTTCTGGCATTCCTATT
TATAGAGTAATGGATAGACAGGGGCAAATAATTAACCCCTCCGAGGATCCACATCTTCCCAAGGAAAAA
GTTCTTAAATTGTATAAGTCTATGACCTTGCTTAACACCATGGACAGGATACTCTATGAATCTCAGAGA
CAGGGCAGGATTAGTTTCTATATGACAAATTATGGAGAAGAAGGAACCCACGTGGGGAGCGCAGCCGCC
TTGGATAACACCGACTTGGTCTTTGGACAGTACAGGGAGGCAGGTGTTCTTATGTACAGGGACTATCCC
CTTGAGCTGTTCATGGCTCAATGTTATGGGAACATTAGTGATCTGGGGAAAGGCCGACAAATGCCCGTG
CATTACGGATGTAAAGAAAGGCATTTTGTAACTATCTCAAGTCCTCTTGCTACTCAAATACCCCAGGCT
GTAGGTGCCGCCTATGCTGCTAAGAGGGCAAACGCCAATAGAGTTGTGATATGCTACTTCGGTGAGGGG
GCTGCAAGCGAGGGAGATGCCCACGCTGGGTTCAACTTTGCAGCCACACTGGAGTGTCCCATTATATTT
TTTTGTAGGAACAATGGCTATGCCATCTCTACCCCAACATCAGAGCAGTACAGAGGAGATGGGATTGCA
GCAAGAGGCCCCGGTTATGGAATCATGTCTATAAGGGTGGATGGGAACGACGTCTTTGCCGTGTATAAC
GCTACTAAAGAGGCCAGAAGAAGGGCTGTGGCTGAGAATCAACCCTTCCTTATAGAGGCCATGACTTAC
AGAATTGGTCATCACTCTACCTCCGATGATTCTTCAGCTTACAGATCCGTGGATGAGGTAAATTACTGG
GATAAACAGGACCATCCTATTTCAAGACTTAGGCATTATCTCCTCAGCCAGGGCTGGTGGGACGAAGAA
CAAGAAAAGGCATGGAGAAAACAATCTAGAAGAAAGGTTATGGAGGCCTTTGAGCAGGCAGAAAGGAAA
CCAAAACCAAATCCCAATCTTCTTTTCTCCGACGTGTACCAGGAAATGCCAGCCCAGCTGAGGAAACAG
CAAGAAAGCCTGGCCAGACATCTTCAGACCTACGGGGAACATTACCCACTGGATCACTTTGACAAATGA
Aagcttggatccaatcaacctctggattacaaaatttgtgaaagattgactggtattcttaactatgtt
gctccttttacgctatgtggatacgctgctttaatgcctttgtatcatgctattgcttcccgtatggct
ttcattttctcctccttgtataaatcctggttgctgtctctttatgaggagttgtggcccgttgtcagg
caacgtggcgtggtgtgcactgtgtttgctgacgcaacccccactggttggggcattgccaccacctgt
cagctcctttccgggactttcgctttccccctccctattgccacggcggaactcatcgccgcctgcctt
gcccgctgctggacaggggctcggctgttgggcactgacaattccgtggtgttgtcggggaaatcatcg
tcctttccttggctgctcgcctgtgttgccacctggattctgcgcgggacgtccttctgctacgtccct
tcggccctcaatccagcggaccttccttcccgcggcctgctgccggctctgcggcctcttccgcgtctt
cgagatctgcctcgactgtgccttctagttgccagccatctgttgtttgcccctcccccgtgccttcct
tgaccctggaaggtgccactcccactgtcctttcctaataaaatgaggaaattgcatcgcattgtctga
gtaggtgtcattctattctggggggtggggtggggcaggacagcaagggggaggattgggaagacaata
gcaggcatgctggggactcgagttaagggcgaattcccgataaggatcttcctagagcatggctacgta
gataagtagcatggcgggttaatcattaactacaaggaacccctagtgatggagttggccactccctct
ctgcgcgctcgctcgctcactgaggccgggcgaccaaaggtcgcccgacgcccgggctttgcccgggcg
gcctcagtgagcgagcgagcgcgcag
SEQ ID NO: 22
> ITR-hAAT-BCKDHB-WT-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagCT
AGCAGGCTCAGAGGCACACAGGAGTTTCTGGGCTCACCCTGCCCCCTTCCAACCCCTCAGTTCCCATCC
TCCAGCAGCTGTTTGTGTGCTGCCTCTGAAGTCCACACTGAACAAACTTCAGCCTACTCATGTCCCTAA
AATGGGCAAACATTGCAAGCAGCAAACAGCAAACACACAGCCCTCCCTGCCTGCTGACCTTGGAGCTGG
GGCAGAGGTCAGAGACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGGATCTTGCTACCAGTGGAACA
GCCACTAAGGATTCTGCAGTGAGAGCAGAGGGCCAGCTAAGTGGTACTCTCCCAGAGACTGTCTGACTC
ACGCCACCCCCTCCACCTTGGACACAGGACGCTGTGGTTTCTGAGCCAGGTACAATGACTCCTTTCGGT
AAGTGCAGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCAGCAG
CCTCCCCCGTTGCCCCTCTGGATCCACTGCTTAAATACGGACGAGGACAGGGCCCTGTCTCCTCAGCTT
CAGGCACCACCACTGACCTGGGACAGTGAATGTAAGTATCAAGGTTACAAGACAGGTTTAAGGAGACCA
ATAGAAACTGGGCTTGTCGAGACAGAGAAGACTCTTGCGTTTCTGATAGGCACCTATTGGTCTTACTGA
CATCCACTTTGCCTTTCTCTCCACAGcaattgcGCGGCCGCCATGGCGGTTGTAGCGGCGGCTGCCGGC
TGGCTACTCAGGCTCAGGGCGGCAGGGGCTGAGGGGCACTGGCGTCGGCTTCCTGGCGCGGGGCTGGCG
CGGGGCTTTTTGCACCCCGCCGCGACTGTCGAGGATGCGGCCCAGAGGCGGCAGGTGGCTCATTTTACT
TTCCAGCCAGATCCGGAGCCCCGGGAGTACGGGCAAACTCAGAAAATGAATCTTTTCCAGTCTGTAACA
AGTGCCTTGGATAACTCATTGGCCAAAGATCCTACTGCAGTAATATTTGGTGAAGATGTTGCCTTTGGT
GGAGTCTTTAGATGCACTGTTGGCTTGCGAGACAAATATGGAAAAGATAGAGTTTTTAATACCCCATTG
TGTGAACAAGGAATTGTTGGATTTGGAATCGGAATTGCGGTCACTGGAGCTACTGCCATTGCGGAAATT
CAGTTTGCAGATTATATTTTCCCTGCATTTGATCAGATTGTTAATGAAGCTGCCAAGTATCGCTATCGC
TCTGGGGATCTTTTTAACTGTGGAAGCCTCACTATCCGGTCCCCTTGGGGCTGTGTTGGTCATGGGGCT
CTCTATCATTCTCAGAGTCCTGAAGCATTTTTTGCCCATTGCCCAGGAATCAAGGTGGTTATACCCAGA
AGCCCTTTCCAGGCCAAAGGACTTCTTTTGTCATGCATAGAGGATAAAAATCCTTGTATATTTTTTGAA
CCTAAAATACTTTACAGGGCAGCAGCGGAAGAAGTCCCTATAGAACCATACAACATCCCACTGTCCCAG
GCCGAAGTCATACAGGAAGGGAGTGATGTTACTCTAGTTGCCTGGGGCACTCAGGTTCATGTGATCCGA
GAGGTAGCTTCCATGGCAAAAGAAAAGCTTGGAGTGTCTTGTGAAGTCATTGATCTGAGGACTATAATA
CCTTGGGATGTGGACACAATTTGTAAGTCTGTGATCAAAACAGGGCGACTGCTAATCAGTCACGAGGCT
CCCTTGACAGGCGGCTTTGCATCGGAAATCAGCTCTACAGTTCAGGAGGAATGTTTCTTGAACCTAGAG
GCTCCTATATCAAGAGTATGTGGTTATGACACACCATTTCCTCACATTTTTGAACCATTCTACATCCCA
GACAAATGGAAGTGTTATGATGCCCTTCGAAAAATGATCAACTATTGAGgatccaatcaacctctggat
tacaaaatttgtgaaagattgactggtattcttaactatgttgctccttttacgctatgtggatacgct
gctttaatgcctttgtatcatgctattgcttcccgtatggctttcattttctcctccttgtataaatcc
tggttgctgtctctttatgaggagttgtggcccgttgtcaggcaacgtggcgtggtgtgcactgtgttt
gctgacgcaacccccactggttggggcattgccaccacctgtcagctcctttccgggactttcgctttc
cccctccctattgccacggcggaactcatcgccgcctgccttgcccgctgctggacaggggctcggctg
ttgggcactgacaattccgtggtgttgtcggggaaatcatcgtcctttccttggctgctcgcctgtgtt
gccacctggattctgcgcgggacgtccttctgctacgtcccttcggccctcaatccagcggaccttcct
tcccgcggcctgctgccggctctgcggcctcttccgcgtcttcgagatctgcctcgactgtgccttcta
gttgccagccatctgttgtttgcccctcccccgtgccttccttgaccctggaaggtgccactcccactg
tcctttcctaataaaatgaggaaattgcatcgcattgtctgagtaggtgtcattctattctggggggtg
gggtggggcaggacagcaagggggaggattgggaagacaatagcaggcatgctggggactcgagttaag
ggcgaattcccgataaggatcttcctagagcatggctacgtagataagtagcatggcgggttaatcatt
aactacaaggaacccctagtgatggagttggccactccctctctgcgcgctcgctcgctcactgaggcc
gggcgaccaaaggtcgcccgacgcccgggctttgcccgggcggcctcagtgagcgagcgagcgcgcag
SEQ ID NO: 23
> ITR-hAAT-BCKDHB-co1-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagCT
AGCAGGCTCAGAGGCACACAGGAGTTTCTGGGCTCACCCTGCCCCCTTCCAACCCCTCAGTTCCCATCC
TCCAGCAGCTGTTTGTGTGCTGCCTCTGAAGTCCACACTGAACAAACTTCAGCCTACTCATGTCCCTAA
AATGGGCAAACATTGCAAGCAGCAAACAGCAAACACACAGCCCTCCCTGCCTGCTGACCTTGGAGCTGG
GGCAGAGGTCAGAGACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGGATCTTGCTACCAGTGGAACA
GCCACTAAGGATTCTGCAGTGAGAGCAGAGGGCCAGCTAAGTGGTACTCTCCCAGAGACTGTCTGACTC
ACGCCACCCCCTCCACCTTGGACACAGGACGCTGTGGTTTCTGAGCCAGGTACAATGACTCCTTTCGGT
AAGTGCAGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCAGCAG
CCTCCCCCGTTGCCCCTCTGGATCCACTGCTTAAATACGGACGAGGACAGGGCCCTGTCTCCTCAGCTT
CAGGCACCACCACTGACCTGGGACAGTGAATGTAAGTATCAAGGTTACAAGACAGGTTTAAGGAGACCA
ATAGAAACTGGGCTTGTCGAGACAGAGAAGACTCTTGCGTTTCTGATAGGCACCTATTGGTCTTACTGA
CATCCACTTTGCCTTTCTCTCCACAGcaattgcGCGGCCGCCATGGCAGTTGTGGCAGCCGCAGCGGGC
TGGTTGTTGCGACTCAGAGCAGCCGGTGCAGAAGGCCATTGGAGACGGTTGCCGGGTGCGGGACTGGCG
CGCGGCTTTCTCCACCCCGCAGCGACTGTAGAAGACGCAGCCCAAAGACGACAGGTCGCTCACTTCACA
TTCCAGCCTGATCCCGAGCCACGAGAATACGGGCAAACGCAAAAAATGAATCTCTTTCAGTCCGTAACA
TCTGCTTTGGATAATAGTCTTGCAAAAGATCCAACAGCTGTAATTTTCGGGGAAGATGTAGCGTTTGGC
GGTGTCTTCCGATGTACCGTCGGGCTGAGGGATAAGTACGGGAAAGATAGAGTATTTAATACCCCCCTG
TGCGAGCAGGGTATAGTCGGATTTGGGATTGGAATAGCCGTAACGGGAGCAACAGCGATTGCCGAAATA
CAATTTGCCGACTATATCTTCCCGGCGTTTGACCAAATTGTTAACGAGGCTGCGAAATATCGGTATCGC
TCCGGCGACTTGTTTAATTGCGGTAGCCTCACAATTAGAAGTCCTTGGGGGTGCGTTGGACACGGTGCG
CTCTATCACAGTCAATCTCCAGAAGCTTTTTTCGCACATTGTCCAGGCATCAAAGTAGTGATTCCCCGA
AGCCCATTTCAGGCGAAAGGTCTCTTGCTCTCCTGTATAGAAGATAAAAACCCATGTATCTTTTTTGAG
CCTAAAATCCTGTACCGCGCCGCAGCTGAGGAAGTCCCTATAGAGCCATACAACATCCCACTCTCACAG
GCAGAAGTTATACAAGAAGGGAGTGACGTGACACTCGTAGCATGGGGGACGCAGGTTCATGTGATCAGA
GAGGTAGCCAGTATGGCAAAAGAGAAATTGGGAGTTTCTTGTGAAGTTATCGATCTCCGAACAATAATC
CCTTGGGATGTAGATACCATTTGTAAGTCTGTTATCAAAACTGGTAGGCTCCTCATATCTCATGAGGCC
CCGTTGACGGGTGGGTTCGCGTCCGAAATTTCATCAACTGTTCAAGAGGAGTGCTTTCTCAACCTGGAA
GCGCCGATCTCTAGAGTCTGCGGATATGATACCCCCTTCCCACACATATTTGAGCCTTTTTATATCCCG
GACAAATGGAAGTGTTACGACGCCCTTCGAAAAATGATAAATTATTGAGgatccaatcaacctctggat
tacaaaatttgtgaaagattgactggtattcttaactatgttgctccttttacgctatgtggatacgct
gctttaatgcctttgtatcatgctattgcttcccgtatggctttcattttctcctccttgtataaatcc
tggttgctgtctctttatgaggagttgtggcccgttgtcaggcaacgtggcgtggtgtgcactgtgttt
gctgacgcaacccccactggttggggcattgccaccacctgtcagctcctttccgggactttcgctttc
cccctccctattgccacggcggaactcatcgccgcctgccttgcccgctgctggacaggggctcggctg
ttgggcactgacaattccgtggtgttgtcggggaaatcatcgtcctttccttggctgctcgcctgtgtt
gccacctggattctgcgcgggacgtccttctgctacgtcccttcggccctcaatccagcggaccttcct
tcccgcggcctgctgccggctctgcggcctcttccgcgtcttcgagatctgcctcgactgtgccttcta
gttgccagccatctgttgtttgcccctcccccgtgccttccttgaccctggaaggtgccactcccactg
tcctttcctaataaaatgaggaaattgcatcgcattgtctgagtaggtgtcattctattctggggggtg
gggtggggcaggacagcaagggggaggattgggaagacaatagcaggcatgctggggactcgagttaag
ggcgaattcccgataaggatcttcctagagcatggctacgtagataagtagcatggcgggttaatcatt
aactacaaggaacccctagtgatggagttggccactccctctctgcgcgctcgctcgctcactgaggcc
gggcgaccaaaggtcgcccgacgcccgggctttgcccgggcggcctcagtgagcgagcgagcgcgcag
SEQ ID NO: 24
> ITR-hAAT-BCKDHB-co2-ITR
ctgcgcgctcgctcgctcactgaggccgcccgggcaaagcccgggcgtcgggcgacctttggtcgcccg
gcctcagtgagcgagcgagcgcgcagagagggagtggccaactccatcactaggggttccttgtagtta
atgattaacccgccatgctacttatctacgtagccatgctctaggaagatcggaattcgcccttaagcT
AGCAGGCTCAGAGGCACACAGGAGTTTCTGGGCTCACCCTGCCCCCTTCCAACCCCTCAGTTCCCATCC
TCCAGCAGCTGTTTGTGTGCTGCCTCTGAAGTCCACACTGAACAAACTTCAGCCTACTCATGTCCCTAA
AATGGGCAAACATTGCAAGCAGCAAACAGCAAACACACAGCCCTCCCTGCCTGCTGACCTTGGAGCTGG
GGCAGAGGTCAGAGACCTCTCTGGGCCCATGCCACCTCCAACATCCACTCGACCCCTTGGAATTTCGGT
GGAGAGGAGCAGAGGTTGTCCTGGCGTGGTTTAGGTAGTGTGAGAGGGGATCTTGCTACCAGTGGAACA
GCCACTAAGGATTCTGCAGTGAGAGCAGAGGGCCAGCTAAGTGGTACTCTCCCAGAGACTGTCTGACTC
ACGCCACCCCCTCCACCTTGGACACAGGACGCTGTGGTTTCTGAGCCAGGTACAATGACTCCTTTCGGT
AAGTGCAGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCAGCAG
CCTCCCCCGTTGCCCCTCTGGATCCACTGCTTAAATACGGACGAGGACAGGGCCCTGTCTCCTCAGCTT
CAGGCACCACCACTGACCTGGGACAGTGAATGTAAGTATCAAGGTTACAAGACAGGTTTAAGGAGACCA
ATAGAAACTGGGCTTGTCGAGACAGAGAAGACTCTTGCGTTTCTGATAGGCACCTATTGGTCTTACTGA
CATCCACTTTGCCTTTCTCTCCACAGcaattgcGCGGCCGCCATGGCAGTTGTGGCAGCTGCAGCAGGC
TGGTTGTTGCGCCTCAGAGCAGCTGGTGCAGAAGGCCATTGGAGAAGGTTGCCTGGTGCCGGACTGGCC
CGCGGCTTTCTCCACCCCGCAGCCACTGTAGAAGATGCAGCCCAAAGAAGACAGGTCGCTCACTTCACA
TTCCAGCCTGATCCCGAGCCAAGAGAATATGGGCAAACCCAAAAAATGAATCTCTTTCAGTCCGTAACA
TCTGCTTTGGATAATAGTCTTGCAAAAGATCCAACAGCTGTAATTTTCGGGGAAGATGTAGCATTTGGA
GGTGTCTTCAGGTGTACAGTCGGGCTGAGGGATAAGTACGGGAAAGATAGAGTATTTAATACCCCCCTG
TGTGAGCAGGGTATAGTGGGATTTGGGATTGGAATAGCTGTAACGGGAGCAACAGCAATTGCTGAAATA
CAATTTGCTGACTATATCTTCCCGGCATTTGACCAAATTGTTAACGAGGCTGCAAAATATAGGTATAGG
TCCGGAGACTTGTTTAATTGTGGTAGCCTCACAATTAGAAGTCCTTGGGGGTGTGTTGGACATGGTGCA
CTCTATCACAGTCAATCTCCAGAAGCTTTTTTCGCACATTGTCCAGGCATCAAAGTAGTGATTCCCAGG
AGCCCATTTCAGGCAAAAGGTCTCTTGCTCTCCTGTATAGAAGATAAAAACCCATGTATCTTTTTTGAG
CCTAAAATCCTGTACAGAGCTGCAGCTGAGGAAGTCCCTATAGAGCCATACAACATCCCACTCTCACAG
GCAGAAGTTATACAAGAAGGGAGTGATGTGACACTGGTAGCATGGGGGACCCAGGTTCATGTGATCAGA
GAGGTAGCCAGTATGGCAAAAGAGAAATTGGGAGTTTCTTGTGAAGTTATCGATCTCCGAACAATAATC
CCTTGGGATGTAGATACCATTTGTAAGTCTGTTATCAAAACTGGTAGGCTCCTCATATCTCATGAGGCC
CCGTTGACCGGTGGGTTCGCATCCGAAATTTCATCAACTGTTCAAGAGGAGTGCTTTCTCAACCTGGAA
GCACCAATCTCTAGAGTCTGTGGATATGATACCCCCTTCCCACACATATTTGAGCCTTTTTATATCCCA
GACAAATGGAAGTGTTACGATGCCCTTAGAAAAATGATAAATTATTGAGgatccaatcaacctctggat
tacaaaatttgtgaaagattgactggtattcttaactatgttgctccttttacgctatgtggatacgct
gctttaatgcctttgtatcatgctattgcttcccgtatggctttcattttctcctccttgtataaatcc
tggttgctgtctctttatgaggagttgtggcccgttgtcaggcaacgtggcgtggtgtgcactgtgttt
gctgacgcaacccccactggttggggcattgccaccacctgtcagctcctttccgggactttcgctttc
cccctccctattgccacggcggaactcatcgccgcctgccttgcccgctgctggacaggggctcggctg
ttgggcactgacaattccgtggtgttgtcggggaaatcatcgtcctttccttggctgctcgcctgtgtt
gccacctggattctgcgcgggacgtccttctgctacgtcccttcggccctcaatccagcggaccttcct
tcccgcggcctgctgccggctctgcggcctcttccgcgtcttcgagatctgcctcgactgtgccttcta
gttgccagccatctgttgtttgcccctcccccgtgccttccttgaccctggaaggtgccactcccactg
tcctttcctaataaaatgaggaaattgcatcgcattgtctgagtaggtgtcattctattctggggggtg
gggtggggcaggacagcaagggggaggattgggaagacaatagcaggcatgctggggactcgagttaag
ggcgaattcccgataaggatcttcctagagcatggctacgtagataagtagcatggcgggttaatcatt
aactacaaggaacccctagtgatggagttggccactccctctctgcgcgctcgctcgctcactgaggcc
gggcgaccaaaggtcgcccgacgcccgggctttgcccgggcggcctcagtgagcgagcgagcgcgcag

In some embodiments, the recombinant nucleic acid molecule of the present invention is inserted in a viral vector, more particularly in an AAV vector.

As used herein the term “AAV” refers to the more than 30 naturally occurring and available adeno-associated viruses, as well as artificial AAVs. Typically the AAV capsid, ITRs, and other selected AAV components described herein, may be readily selected from among any AAV, including, without limitation, AAV1, AAV2, AAV3, AAV4, AAV5, AAV6, AAV6.2, AAV7, AAV8, AAV9, rh10, AAVrh64R1, AAVrh64R2, rh8, variants of any of the known or mentioned AAVs or AAVs yet to be discovered or variants or mixtures thereof. See, e.g., WO 2005/033321. The genomic and protein sequences of various serotypes of AAV, as well as the sequences of the native terminal repeats (TRs), Rep proteins, and capsid subunits including VP1 protein are known in the art. Such sequences may be found in the literature or in public databases such as GenBank or Protein Data Bank (PDB). See, e.g., GenBank and PDB Accession Numbers NC_002077 and 3NG9 (AAV-1), AF043303 and 1LP3 (AAV-2), NC_001729 (AAV-3), U89790 and 2G8G (AAV-4), NC_006152 and 3NTT (AAV-5), 3OAH (AAV6), AF513851 (AAV-7), NC_006261 and 2QA0 (AAV-8), AY530579 and 3UX1 (AAV-9 (isolate hu.14)); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; and U.S. Pat. Nos. 6,156,303 and 7,906,111.

In some embodiments, the AAV vector is used in association with exosomes (exo-AAV) as described in WO2017136764 and in Hudry, Eloise, et al. “Exosome-associated AAV vector as a robust and convenient neuroscience tool.” Gene therapy 23.4 (2016): 380.

In some embodiments, the recombinant nucleic acid molecule of the present invention is inserted in a recombinant AAV8 viral particle.

As used herein, the term “recombinant AAV8 viral particle” refers to a viral particle that has an AAV8 capsid, the capsid having packaged therein the expression cassette comprising the recombinant nucleic molecule of the present invention.

As used herein, “AAV8 capsid” refers to the AAV8 capsid having the encoded amino acid sequence of GenBank accession:YP_077180, which is incorporated by reference herein and reproduced in SEQ ID NO:25.

SEQ ID NO: 25
> capsid protein [Adeno-associated virus - 8]
MAADGYLPDW LEDNLSEGIR EWWALKPGAP KPKANQQKQD
DGRGLVLPGY KYLGPFNGLD KGEPVNAADA AALEHDKAYD
QQLQAGDNPY LRYNHADAEF QERLQEDTSF GGNLGRAVFQ
AKKRVLEPLG LVEEGAKTAP GKKRPVEPSP QRSPDSSTGI
GKKGQQPARK RLNFGQTGDS ESVPDPQPLG EPPAAPSGVG
PNTMAAGGGA PMADNNEGAD GVGSSSGNWH CDSTWLGDRV
ITTSTRTWAL PTYNNHLYKQ ISNGTSGGAT NDNTYFGYST
PWGYFDFNRF HCHFSPRDWQ RLINNNWGFR PKRLSFKLFN
IQVKEVTQNE GTKTIANNLT STIQVFTDSE YQLPYVLGSA
HQGCLPPFPA DVFMIPQYGY LTLNNGSQAV GRSSFYCLEY
FPSQMLRTGN NFQFTYTFED VPFHSSYAHS QSLDRLMNPL
IDQYLYYLSR TQTTGGTANT QTLGFSQGGP NTMANQAKNW
LPGPCYRQQR VSTTTGQNNN SNFAWTAGTK YHLNGRNSLA
NPGIAMATHK DDEERFFPSN GILIFGKQNA ARDNADYSDV
MLTSEEEIKT TNPVATEEYG IVADNLQQQN TAPQIGTVNS
QGALPGMVWQ NRDVYLQGPI WAKIPHTDGN FHPSPLMGGF
GLKHPPPQIL IKNTPVPADP PTTFNQSKLN SFITQYSTGQ
VSVEIEWELQ KENSKRWNPE IQYTSNYYKS TSVDFAVNTE
GVYSEPRPIG TRYLTRNL

The expression cassette of the recombinant AAV8 viral particle typically contains an AAV2 inverted terminal repeat sequence flanking the recombinant nucleic acid molecule of the present invention, in which the transgene sequence is operably linked to expression control sequences. Such a rAAV viral particle is termed “pharmacologically active” when it delivers the transgene to a host cell which is capable of expressing the desired gene product carried by the expression cassette.

Numerous methods are known in the art for production of rAAV vectors, including transfection, stable cell line production, and infectious hybrid virus production systems which include Adenovirus-AAV hybrids, herpesvirus-AAV hybrids and baculovirus-AAV hybrids. See, e.g., G Ye, et al, Hu Gene Ther Clin Dev, 25: 212-217 (December 2014); R M Kotin, Hu Mol Genet, 2011, Vol. 20, Rev Issue 1, R2-R6; M. Mietzsch, et al, Hum Gene Therapy, 25: 212-222 (March 2014); T Virag et al, Hu Gene Therapy, 20: 807-817 (August 2009); N. Clement et al, Hum Gene Therapy, 20: 796-806 (August 2009); DL Thomas et al, Hum Gene Ther, 20: 861-870 (August 2009). rAAV production cultures for the production of rAAV virus particles may require; 1) suitable host cells, including, for example, human-derived cell lines such as HeLa, A549, or 293 cells, or insect-derived cell lines such as SF-9, in the case of baculovirus production systems; 2) suitable helper virus function, provided by wild type or mutant adenovirus (such as temperature sensitive adenovirus), herpes virus, baculovirus, or a nucleic acid construct providing helper functions in trans or in cis; 3) functional AAV rep genes, functional cap genes and gene products; 4) a transgene (such as a therapeutic transgene) flanked by AAV ITR sequences; and 5) suitable media and media components to support rAAV production. A variety of suitable cells and cell lines have been described for use in production of AAV. The cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. rAAV vector particles may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases. In some embodiments, the rAAV production culture harvest is clarified to remove host cell debris. Suitably, the rAAV production culture harvest is treated with a nuclease, or a combination of nucleases, to digest any contaminating high molecular weight nucleic acid present in the production culture. The mixture containing full rAAV particles may be isolated or purified using one or more of the following purification steps: tangential flow filtration (TFF) for concentrating the rAAV particles, heat inactivation of helper virus, rAAV capture by hydrophobic interaction chromatography, buffer exchange by size exclusion chromatography (SEC), and/or nanofiltration. These steps may be used alone, in various combinations, or in different orders.

The recombinant AAV8 viral particle of the present invention is particularly suitable for the treatment of maple syrup urine disease (MSUD).

Therefore, a further object of the present invention relates to a method of treating MSUD in a subject in need thereof comprising administering to the subject a therapeutically effective amount of the recombinant AAV8 viral particle of the present invention.

As used herein, the term “maple syrup urine disease” or “MSUD” has its general meaning in the art and refers to an inherited disorder in which the body is unable to process certain protein building blocks (amino acids) properly. The condition gets its name from the distinctive sweet odor of affected infants' urine. It is also characterized by poor feeding, vomiting, lack of energy (lethargy), abnormal movements, and delayed development. If untreated, maple syrup urine disease can lead to seizures, coma, and death. Maple syrup urine disease is often classified by its pattern of signs and symptoms. The most common and severe form of the disease is the classic type, which becomes apparent soon after birth. Variant forms of the disorder become apparent later in infancy or childhood and are typically milder, but they still lead to delayed development and other health problems if not treated.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By a “therapeutically effective amount” is meant a sufficient amount of cells generated with the present invention for the treatment of the disease at a reasonable benefit/risk ratio applicable to any medical treatment. It will be understood that the total usage of these cells will be decided by the attending physicians within the scope of sound medical judgment. The specific therapeutically effective dose level for any particular subject will depend upon a variety of factors including the age, body weight, general health, sex and diet of the subject; the time of administration, route of administration, and survival rate of the cells employed; the duration of the treatment; drugs used in combination or coincidental with the administered cells; and like factors well known in the medical arts. For example, it is well known within the skill of the art to start doses of cells at levels lower than those required to achieve the desired therapeutic effect and to gradually increase the dosage until the desired effect is achieved.

By a “therapeutically effective amount” is meant a sufficient amount of the vector to treat the maple syrup urine disease at a reasonable benefit/risk ratio. It will be understood that the total daily usage of the vector will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. Thus, the doses of vectors may be adapted depending on the disease condition, the subject (for example, according to his weight, metabolism, etc.), the treatment schedule, etc. Typically, the doses of AAV vectors to be administered in humans may range from 5.10¹¹ to 5.10¹⁴ vg/kg.

In some embodiments, the recombinant AAV8 viral particle of the present invention is administered to the subject intravenously.

The recombinant AAV8 viral particle of the present invention is thus formulated into pharmaceutical compositions. These compositions may comprise, in addition to the vector, a pharmaceutically acceptable excipient, carrier, buffer, stabiliser or other materials well known to those skilled in the art. Such materials should be non-toxic and should not interfere with the efficacy of the active ingredient (i.e. the vector of the invention). The precise nature of the carrier or other material may be determined by the skilled person according to the route of administration, i.e. here intravitreal injection. The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. For injection, the active ingredient will be in the form of an aqueous solution, which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required. For delayed release, the vector may be included in a pharmaceutical composition, which is formulated for slow release, such as in microcapsules formed from biocompatible polymers or in liposomal carrier systems according to methods known in the art. Typically, the pharmaceutical composition of the present invention is supplied in a prefilled syringe. A “ready-to-use syringe” or “prefilled syringe” is a syringe which is supplied in a filled state, i.e. the pharmaceutical composition to be administered is already present in the syringe and ready for administration. Prefilled syringes have many benefits compared to separately provided syringe and vial, such as improved convenience, affordability, accuracy, sterility, and safety. The use of prefilled syringes results in greater dose precision, in a reduction of the potential for needle sticks injuries that can occur while drawing medication from vials, in pre-measured dosage reducing dosing errors due to the need to reconstituting and/or drawing medication into a syringe, and in less overfilling of the syringe helping to reduce costs by minimising drug waste. In some embodiments the pH of the liquid pharmaceutical composition of the present invention is in the range of 5.0 to 7.0, 5.1 to 6.9, 5.2 to 6.8, 5.3 to 6.7 or 5.4 to 6.6.

The invention will be further illustrated by the following figures and examples. However, these examples and figures should not be interpreted in any way as limiting the scope of the present invention.

### FIGURES

FIG. 1. Bckdha^{-/-} mouse model recapitulates the severe human MSUD phenotype. a) Kaplan-Meier curves showing the survival probability during the first 30 days of life (Bckdha^{-/-} N=15, Bckdha^{+/-} N=44, Bckdha^{+/+} N=26); **** P<0.0001 for the comparison Bckdha^{-/-} vs Bckdha^{+/+} and Bckdha^{-/-} vs Bckdha^{+/-}, log-rank Mantel-Cox test. b) Weight curves showing major growth delay for Bckdha^{-/-} (Bckdha^{-/-} N=1, Bckdha^{+/-} N=2, Bckdha^{+/+} N=3); data are means±SD. c) Weights at 1 week; data are means±SD; **** P<0.0001 for the comparison Bckdha^{-/-} vs Bckdha^{+/+} and Bckdha^{-/-} vs Bckdha^{+/-}, one-way analysis of variance (ANOVA) with Tukey's post hoc. d) Leucine concentrations in plasma at 3 days; data are means±SD; **** P<0.0001 for the comparison Bckdha^{-/-} vs Bckdha^{+/+} and Bckdha^{+/-} vs Bckdha^{+/-}, one-way analysis of variance (ANOVA) with Tukey's post hoc. e) Alloisoleucine concentrations in plasma at 3 days; data are means±SD.

FIG. 2. Scheme of the optimised AAV expression cassettes coding for either human BCKDHA or BCKDHB with (a) the Human elongation factor-1 alpha promoter (EF-1 alpha) and (b) the Human alpha anti-trypsin promoter (hAAT).

FIG. 3. High dose gene therapy allows long-term rescue of severe MSUD phenotype of Bckdha−/− mice with EF1α hBCKDHA transgene. a) Weight curves for males (Bckdha^{-/-} N=6, Bckdha−/− N=3, Bckdha^{+/+} N=3); data are means±SD. b) Weight curves for females (Bckdha^{-/-} N=3, Bckdha^{+/-} N=8, Bckdha^{+/+} N=6); data are means±SD. c) Leucine concentrations in plasma ((Bckdha^{-/-} non injected N=3, Bckdha^{-/-} injected N=5, Bckdha^{+/-} injected N=5, Bckdha^{+/+} injected N=5); data are means±SD; Western blot analyses of Bckdha^{-/-} mice sacrificed at 6 months (N=5) and 1 non injected Bckdha^{-/-} mice died at 1 week and 1 non injected Bckdha^{+/+}. Histograms represent 3 technical replicates per individual. Data are means and SEM. d) Liver. e) Heart. f) Brain. g) Muscle.

FIG. 4. Reducing EF1a hBCKDHA dosage allows partial albeit transient rescue of the MSUD phenotype in Bckdha−/− mice. a) Weight curves (Bckdha^{-/-} injected with 10¹³ vg/kg and sacrificed <4 weeks N=2, Bckdha^{-/-} injected with 10¹³ vg/kg and sacrificed at 4 weeks N=5, Bckdha^{-/-} injected with 10¹⁴ vg/kg and sacrificed at 4 weeks N=2, Bckdha^{+/-} N=18, Bckdha^{+/+} N=9); data are means±SD. b) Leucine concentrations in plasma at sacrifice (Bckdha^{-/-} injected with 10¹³ vg/kg and sacrificed 4 weeks N=2, Bckdha^{-/-} injected with 10¹³ vg/kg and sacrificed at 4 weeks N=5, Bckdha^{-/-} injected with 10¹⁴ vg/kg and sacrificed at 4 weeks N=2, Bckdha^{+/-} N=18, Bckdha^{+/+} N=9) or at 4 weeks (Bckdha^{-/-} injected with 10¹³ vg/kg from the previous experiment N=3); data are means±SD. Western blot analyses of Bckdha^{-/-} mice injected with 10¹³ vg/kg and sacrificed at 4 weeks (N=7), Bckdha^{-/-} mice injected with 10¹⁴ vg/kg and sacrificed at 4 weeks (N=2) and 1 non injected Bckdha^{-/-} mice died at 1 week and 1 non injected Bckdha^{+/+}. Histograms represent 3 technical replicates per individual. Data are means and SEM. c) Liver. d) Heart. e) Brain. f) Muscle.

FIG. 5. Gene therapy with hAAT hBCKDHA transgene allows transient rescue of the MSUD phenotype in Bckdha^{-/-} mice. Weight curves showing a growth arrest and weight loss after day 14 leading to sacrifice at day 19 or 21 (Bckdha^{-/-} with initial normal growth N=3, Bckdha^{-/-} with growth on a lower curve N=2, Bckdha^{+/-} N=10, Bckdha^{+/+} N=6); data are means±SD.

FIG. 6. Bckdhb^{-/-} mouse model recapitulates the severe human MSUD phenotype. a) Kaplan-Meier curves showing the survival probability during the first 16 days of life (Bckdhb^{-/-} N=7, Bckdhb^{+/+} N=8, Bckdhb^{+/+} N=4). b) Leucine and c) alloisoleucine concentrations in plasma at 1 days (Bckdhb^{-/-} N=7, Bckdhb^{+/-} N=8, Bckdhb^{+/+} N=4). Data are means±SD.

FIG. 7. High dose gene therapy allows rescue of severe MSUD phenotype of Bckdhb^{-/-} mice with EF1α hBCKDHB transgene. Weight curves for a) males (Bckdhb^{-/-} N=5, Bckdhb^{+/-} N=4) and b) females (Bckdhb^{-/-} N=2, Bckdhb^{+/-} N=4, Bckdhb^{+/+} N=1). c) Leucine concentrations in plasma at 28 and 56 days post injection (Bckdha^{-/-} N=7, Bckdha^{+/-} N=8, Bckdha^{+/+} N=1). Data are means±SD.

### EXAMPLE

### Example 1

Bckdha−/− Mice Recapitulate the Severe Human MSUD Phenotype

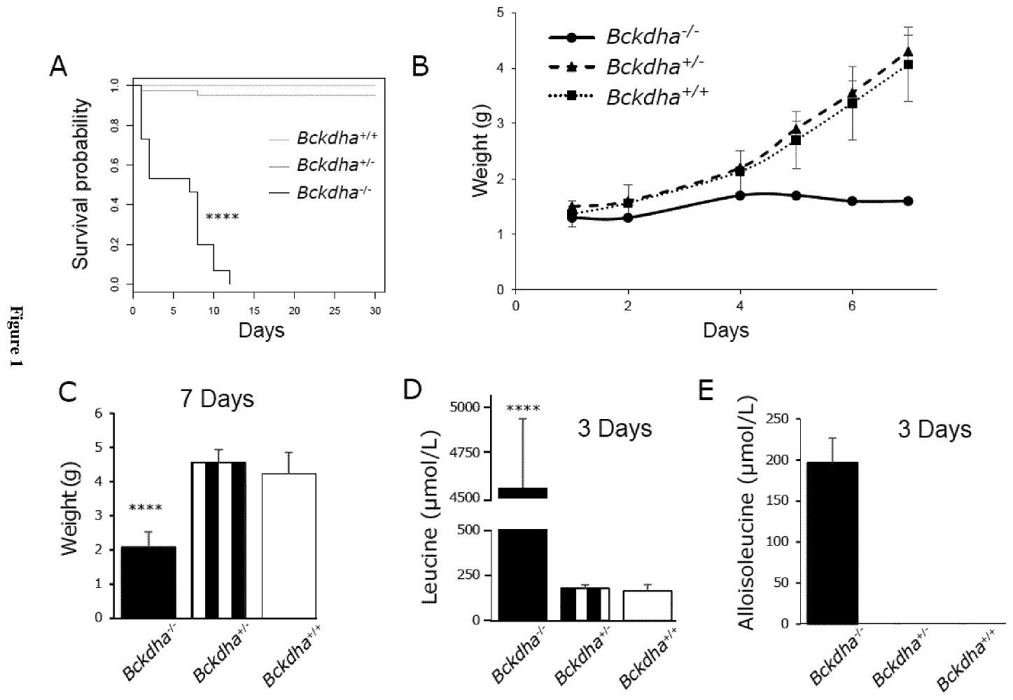

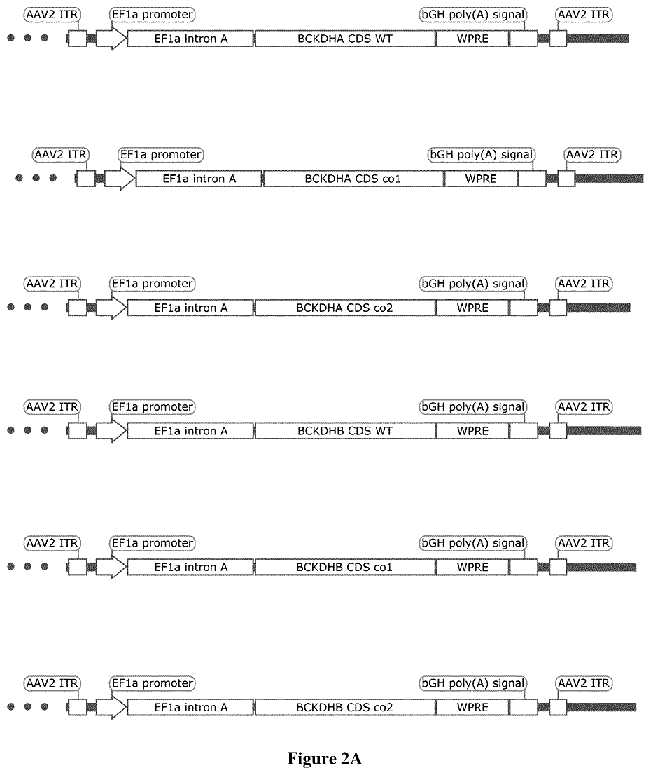

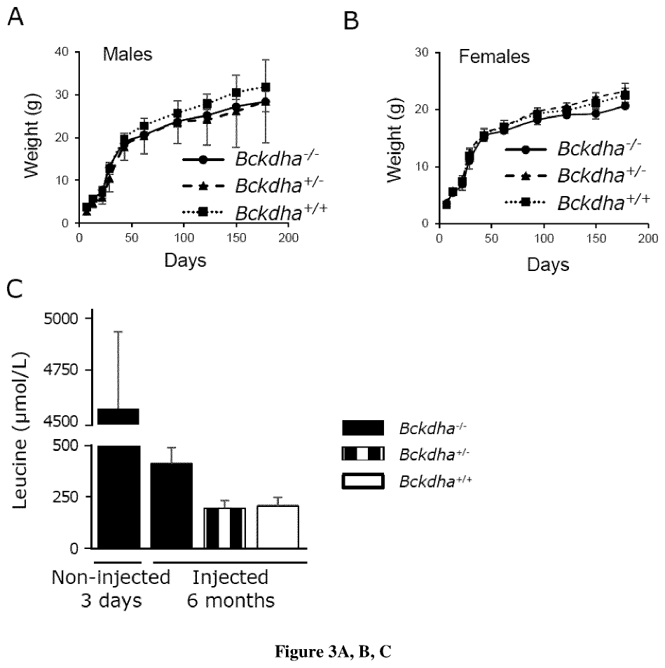

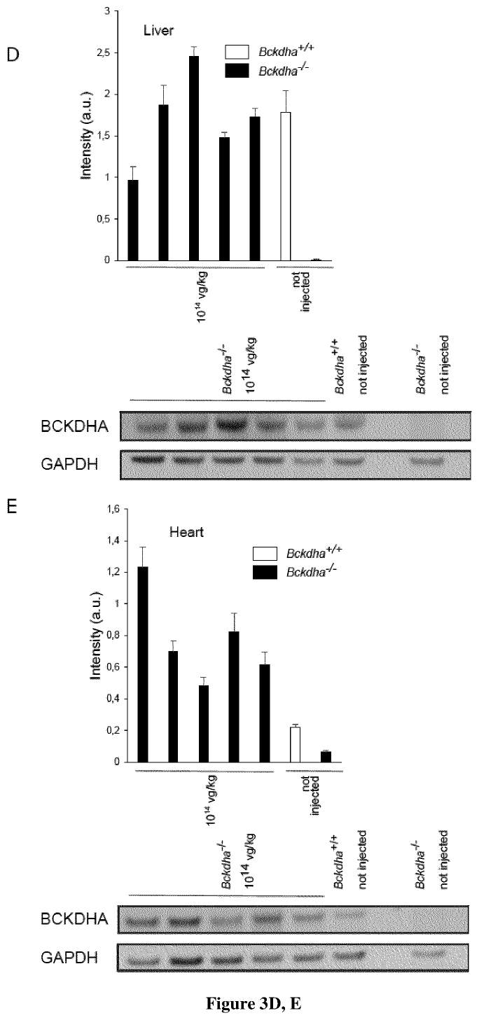

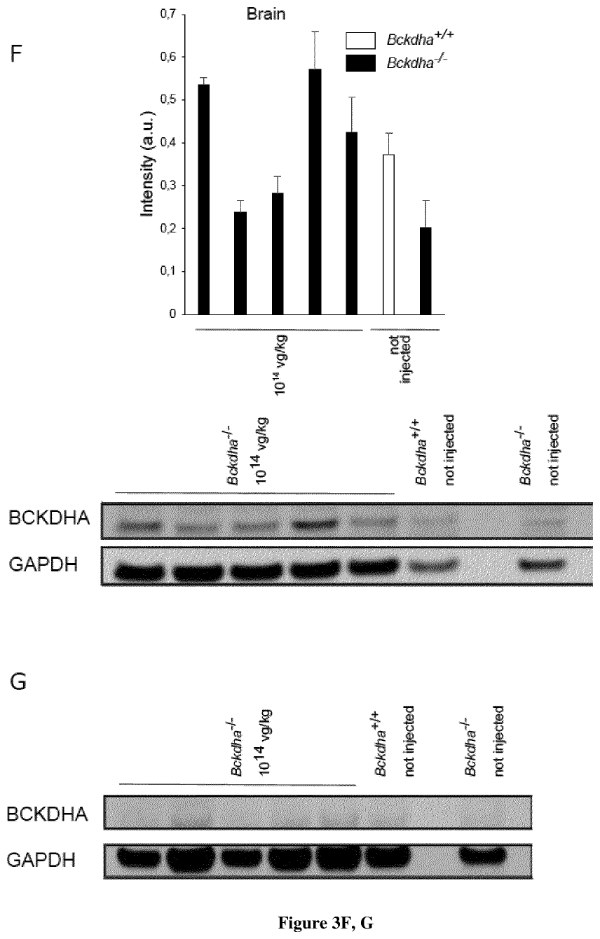

We generated Bckdha−/− mice by crossing commercial heterozygous Bckdha+/− males and females, which did not display any particular phenotype. Bckdha−/− mice showed a lethal early-onset phenotype. Fifty percent of mice died before P3, 50% around P7 with a maximum life expectancy of 12 days (FIG. 1a). Bckdha−/− mice had a major growth delay (FIG. 1b, c). Mice that survived for more than a week showed reduced activity and abnormal response in the hindlimb test. At the biochemical level, Bckdha−/− mice displayed a major increase of branched-chain amino acids (FIG. 1d) and accumulation of alloisoleucine, a pathognomonic marker of MSUD in humans (FIG. 1e) in blood. In wild-type (WT) individuals, western blot showed that Bckdha protein was not expressed in skeletal muscle (quadriceps) (FIG. 3g) and showed mild expression in brain (FIG. 3f). In Bckdha−/− mice, Bckdha protein was absent in liver and heart, confirming the null nature of the model (FIG. 3d, e).

Design and In Vitro Validation of a Viral Vector for the Treatment of MSUD

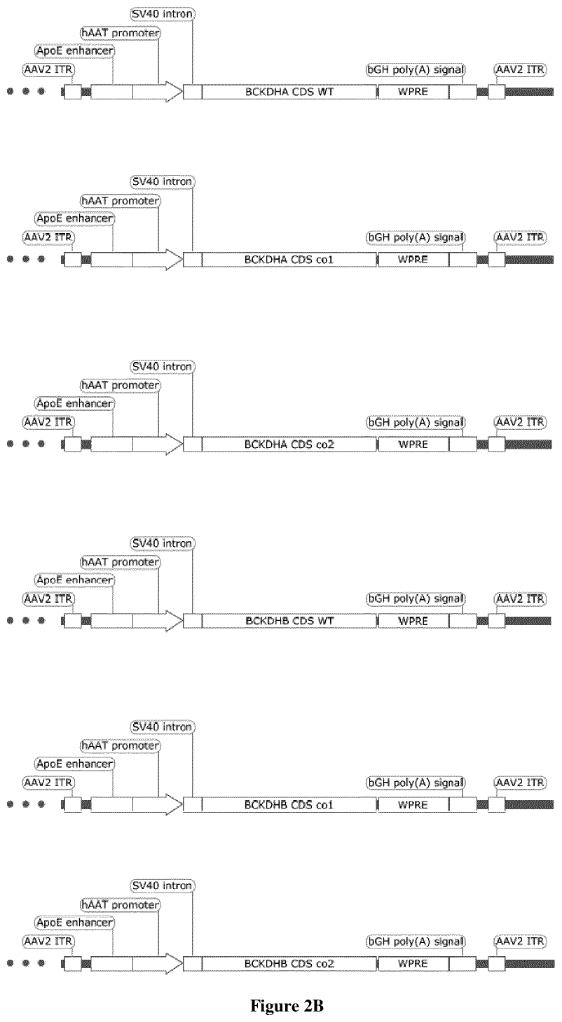

To maximize transgene expression in liver in vivo we developed optimised AAV expression cassettes coding for either human BCKDHA or BCKDHB. We generated 3 variants of each gene coding sequence (CDS): the wild-type version (WT) and 2 different codon-optimised versions, the first one denominated co1 is a classic optimisation to increase protein expression and the second one, denominated co2 has a reduced CpG content (FIG. 2a, b). This is due to the fact that the reduction or elimination of immunostimulatory CpG sequences in plasmid expression vectors prevents the stimulation of transgene product-specific immune responses without necessarily reducing transgene expression.

The capsid serotype of choice was AAV8 due to its tropism to the liver; two different promoters, one ubiquitous, the Human elongation factor-1 alpha promoter (EF-1 alpha) (FIG. 2a) and one liver specific, the Human alpha anti-trypsin promoter (hAAT) were chosen to compare the protein expression (FIG. 2b), vector genome copy number (VGCN) in different tissues along with mRNA expression.

Intravenous EF1a hBCKDHA Allows Long-Term and Sustainable Rescue of Severe MSUD phenotype of Bckdha−/− Mice

In order to establish a proof of concept of treatment efficacy, we performed systemic intra-temporal injection of hBCKDHA transgene under the control of the ubiquitous promoter EF1α encapsulated in AAV8 at 10¹⁴ vg/kg (further referred to as high dose) at P0, immediately after birth in mice pups. All the pups of the litters were injected, prior to get the genotype results. One pup died at P2 without corpse for genotyping and was not further included in the study. Genotypes were performed at P10. Nine Bckdha−/− pups from 3 litters were injected. Compared to their wild-type and heterozygous littermates they exhibited similar survival and a normal growth (FIG. 3a, b). At age 6 months these 9 pups were still alive without overt phenotypic abnormalities (FIG. 3a, b). The biochemical phenotype was dramatically improved (FIG. 3c). We sacrificed 5 Bckdha−/− mice at 6 months of age. At that age, hBCKDHA protein was detectable mainly in the liver and the heart and present albeit at lower levels in brain and skeletal muscle (FIG. 3d, e, f, g). The remaining 4 Bckdha−/− mice were still alive without overt phenotypic abnormalities at age 12 months.

Reducing EF1a hBCKDHA Dosage Allows Partial Though Transient Rescue of the MSUD Phenotype in Bckdha−/− Mice

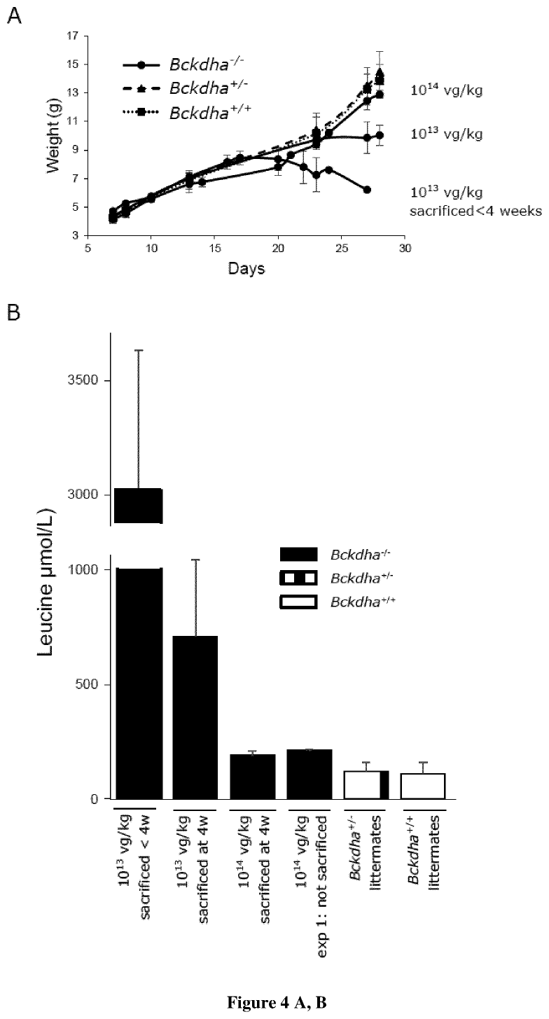

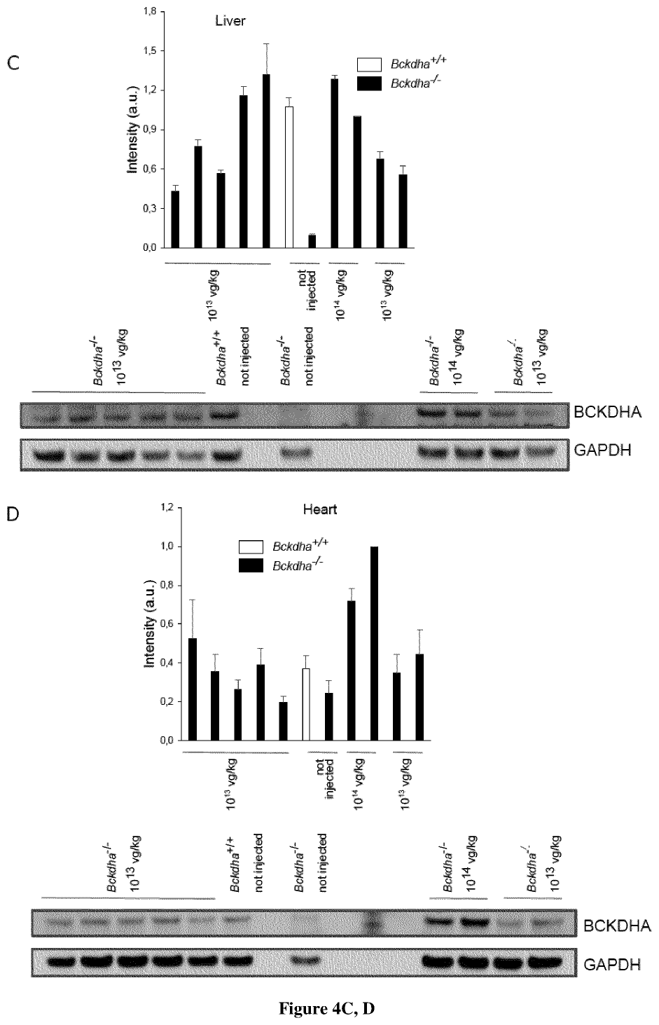

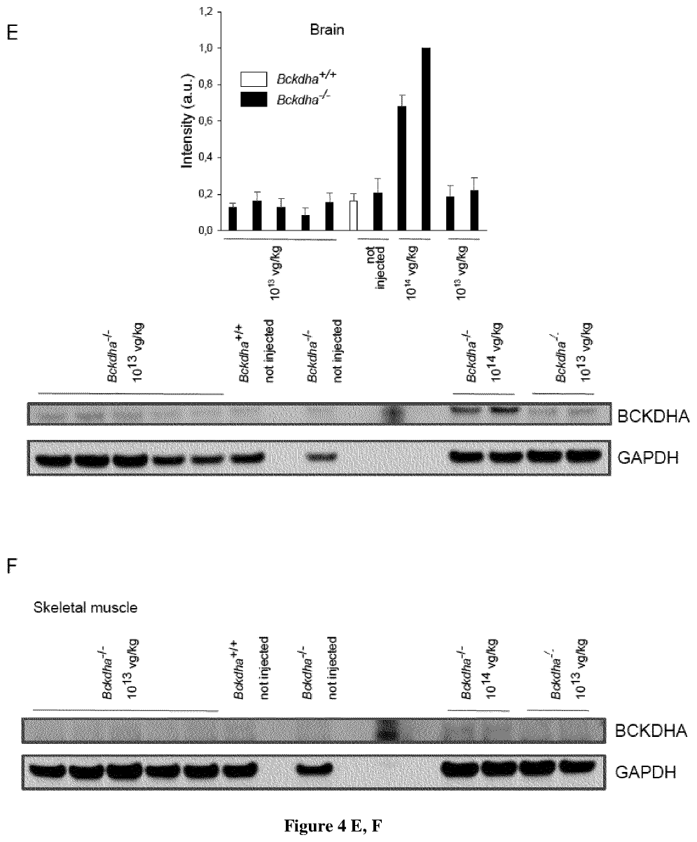

We performed the same experiment reducing EF1α hBCKDHA in mice to 10¹³ vg/kg. Three litters were injected at P0 with EF1α hBCKDHA at 10¹³ vg/kg and two as control with EF1α hBCKDHA at 10¹⁴ vg/kg. In the litters injected at 10¹³ vg/kg, one pup died at P3 without corpse, one Bckdha^{-/-} died at P1 probably due to injection failure and one Bckdha^{-/-} died at P7 of traumatic urine sampling, leaving for the analysis 7 Bckdha^{-/-}, 9 Bckdha^{+/-} and 5 Bckdha^{+/+} mice. In the litters injected at 10¹⁴ vg/kg, one Bckdha^{-/-} died at P2 probably due to injection failure and one Bckdha^{+/-} died at P7 in a context of major growth retardation, leaving for the analysis 2 Bckdha^{-/-}, 9 Bckdha^{+/-} and 4 Bckdha^{+/+} mice. With the injections at 10¹³ vg/kg, we observed a partial and transient recue of the MSUD phenotype in Bckdha−/− mice (N=7) with important inter-individual variability. Five out of seven Bckdha−/− mice showed a normal growth without obvious neurological signs during the first 3 weeks but then stopped growing and developed neurological signs (chiefly ataxia with frequent falls), urging us to sacrifice them at age 4 weeks (FIG. 4a). Two out of seven Bckdha−/− mice showed a more severe evolution, with a growth arrest during the third week followed by weight loss and the development of neurological signs evolving towards a moribund state requiring an anticipated sacrifice before age 4 weeks (FIG. 4a). As expected, the Bckdha−/− mice injected with EF1α hBCKDHA at 10¹⁴ vg/kg displayed a normal growth without any neurological signs (FIG. 4a N=2 Bckdha−/− injected at 10¹⁴ vg/kg and sacrificed at 4 weeks). We observed a similar dose effect at the biochemical level: while the Bckdha−/− mice injected at 10¹⁴ vg/kg displayed normal-high plasma leucine concentrations, the Bckdha−/− mice injected at 10¹³ vg/kg and sacrificed at 4 weeks displayed a marked increase in leucine concentrations (FIG. 4b). The two Bckdha−/− mice injected at 10¹³ vg/kg and sacrificed before 4 weeks showed a massive increase in leucine concentrations consistent with their worse clinical state, suggesting a loss of treatment efficacy and a relapse of the disease. Western blot analysis showed an important increase of hBCKDHA expression in liver for the two dosages, an increase of hBCKDHA expression in heart with a dose effect and a detectable hBCKDHA expression only at the 10¹⁴ vg/kg dosage in the brain (FIG. 4c, d, e, f).

Intravenous hAAT hBCKDHA Allows Transient Rescue of the MSUD Phenotype in Bckdha^{-/-} Mice

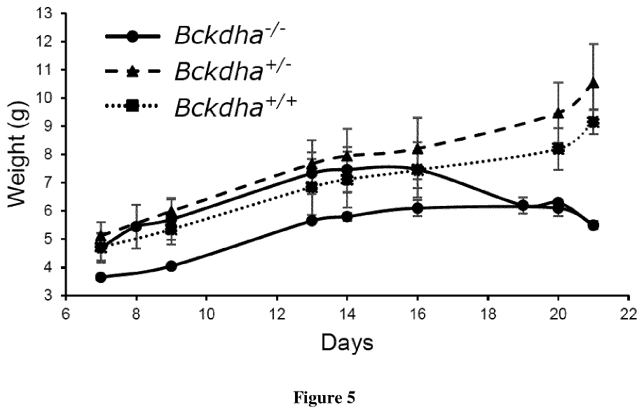

To evaluate the contribution of liver and extra-hepatic tissues to the whole-body BCKDHA enzyme activity responsible for the phenotypic rescue of mice treated with the EF1α hBCKDHA transgene at 10¹⁴ vg/kg, we tested a non-ubiquitous liver-specific promotor (hAAT) with a dosage of 10¹³ vg/kg that would be equivalent to 10¹⁴ vg/kg with EF1α in terms of “liver” targeting. We performed systemic intra-temporal injections at P0, immediately after birth in three litters. One Bckdha^{-/-} died at P1, probably due to injection failure and one Bckdha^{+/-} died at P12 with a major growth retardation and was not included in the analysis, leaving 5 Bckdha^{-/-}, 10 Bckdha^{+/-} and 6 Bckdha^{+/+} mice. This treatment allowed a transient rescue of the MSUD phenotype as 5/5 Bckdha^{-/-} mice survived more than 14 days without overt clinical symptoms. 3/5 Bckdha^{-/-} mice exhibited a strictly normal growth until P14, followed by a rapid weight loss, appearance of clinical signs (ataxia, frequent falls) evolving towards a moribund state requiring sacrifice at P19 or P21 (FIG. 5). The 2 last Bckdha^{-/-} mice displayed growth on a lower curve during the 2 first weeks followed by a growth arrest during the third week with neurological deterioration and weight loss at P20 requiring sacrifice at P21. These results suggest that of non-liver tissues effectively contribute to the whole-body BCKDHA activity responsible for the phenotypic rescue of mice treated with the EF1α hBCKDHA transgene at 10¹⁴ vg/kg.

### Example 2

Bckdhb^{-/-} Mouse Model Recapitulates the Severe Human MSUD Phenotype

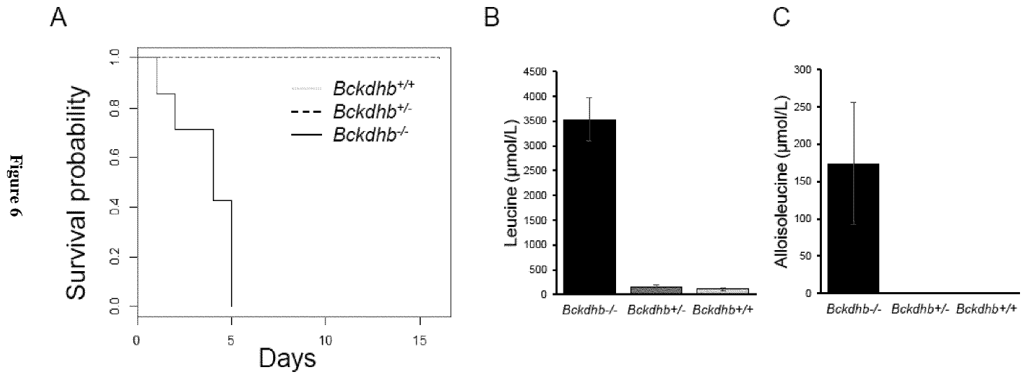

Bckdhb^{-/-} mouse model recapitulates the severe human MSUD phenotype, displaying a lethal early phenotype (FIG. 6a) with major accumulation of MSUD markers, leucine (FIG. 6b) and alloisoleucine, in plasma (FIG. 6c).

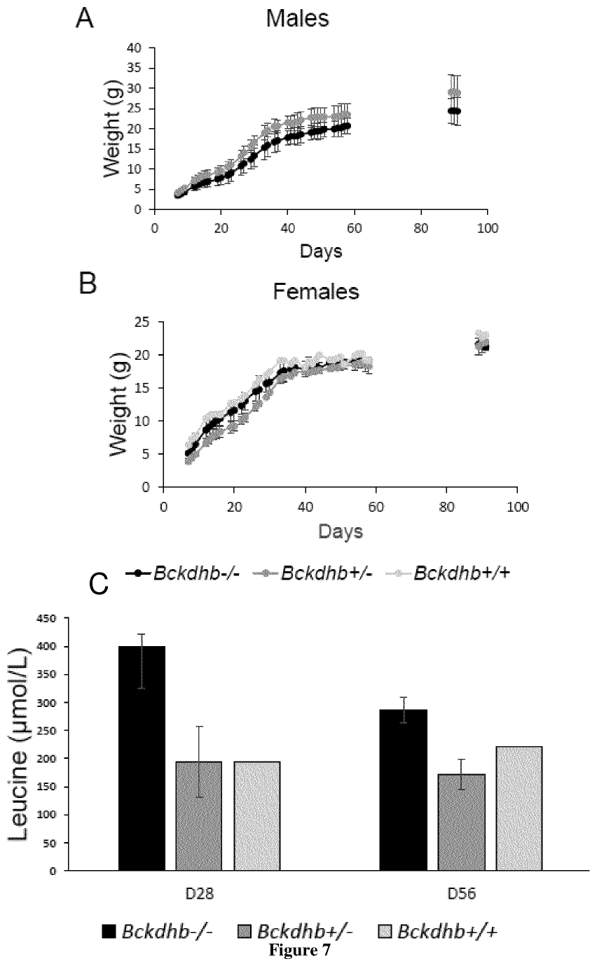

High dose gene therapy allows rescue of severe MSUD phenotype of Bckdhb^{-/-} mice with EF1α hBCKDHB transgene. In order to establish a proof of concept of treatment efficacy, we performed systemic intra-temporal injection of hBCKDHB transgene under the control of the ubiquitous promoter EF1a encapsulated in AAV8 at 10¹⁴ vg/kg at P0, immediately after birth in mice pups. Compared to their wild-type and heterozygous littermates Bckdhb^{-/-} mice exhibited similar survival and a normal growth (FIG. 7a, b) without overt phenotypic abnormalities at age 3 months, with a dramatic improvement of the biochemical phenotype (FIG. 7c).

### REFERENCES

Throughout this application, various references describe the state of the art to which this invention pertains. The disclosures of these references are hereby incorporated by reference into the present disclosure.
Abi-Wardé, M.-T., Roda, C., Arnoux, J.-B., Servais, A., Habarou, F., Brassier, A., Pontoizeau, C., Barbier, V., Bayart, M., Leboeuf, V., et al. (2017). Long-term metabolic follow-up and clinical outcome of 35 patients with maple syrup urine disease. J. Inherit. Metab. Dis. 40, 783-792.
Baruteau, J Perocheau, D. P., Hanley, J Lorvellec, M., Rocha-Ferreira, E., Karda, R., Ng, J Suff, N., Diaz, J. A., Rahim, A. A., et al. (2018). Argininosuccinic aciduria fosters neuronal nitrosative stress reversed by Asl gene transfer. Nat. Commun. 9, 3505.
Bodner-Leidecker, A., Wendel, U., Saudubray, J. M., and Schadewaldt, P. (2000). Branched-chain L-amino acid metabolism in classical maple syrup urine disease after orthotopic liver transplantation. J. Inherit. Metab. Dis. 23, 805-818.
Bouchereau, J., Leduc-Leballeur, J., Pichard, S., Imbard, A., Benoist, J.-F., Abi Warde, M.-T., Arnoux, J.-B., Barbier, V., Brassier, A., Broué, P., et al. (2017). Neurocognitive profiles in MSUD school-age patients. J. Inherit. Metab. Dis. 40, 377-383.
Chandler, R. J., and Venditti, C. P. (2019). Gene Therapy for Methylmalonic Acidemia: Past, Present, and Future. Hum. Gene Ther.
Chandler, R. J., Tarasenko, T. N., Cusmano-Ozog, K., Sun, Q., Sutton, V. R., Venditti, C. P., and McGuire, P. J. (2013). Liver-directed adeno-associated virus serotype 8 gene transfer rescues a lethal murine model of citrullinemia type 1. Gene Ther. 20, 1188-1191.
Cunningham, S. C., Spinoulas, A., Carpenter, K. H., Wilcken, B., Kuchel, P. W., and Alexander, I. E. (2009). AAV2/8-mediated correction of OTC deficiency is robust in adult but not neonatal Spf(ash) mice. Mol. Ther. J. Am. Soc. Gene Ther. 17, 1340-1346.
Ginocchio, V. M., Ferla, R., Auricchio, A., and Brunetti-Pierri, N. (2019). Current Status on Clinical Development of Adeno-Associated Virus-Mediated Liver-Directed Gene Therapy for Inborn Errors of Metabolism. Hum. Gene Ther.
Grisch-Chan, H. M., Schwank, G., Harding, C. O., and Thöny, B. (2019). State-of-the-Art 2019 on Gene Therapy for Phenylketonuria. Hum. Gene Ther.
Homanics, G. E., Skvorak, K., Ferguson, C., Watkins, S., and Paul, H. S. (2006). Production and characterization of murine models of classic and intermediate maple syrup urine disease. BMC Med. Genet. 7, 33.
Lee, E. K., Hu, C., Bhargava, R., Rozengurt, N., Stout, D., Grody, W. W., Cederbaum, S. D., and Lipshutz, G. S. (2012). Long-term survival of the juvenile lethal arginase-deficient mouse with AAV gene therapy. Mol. Ther. J. Am. Soc. Gene Ther. 20, 1844-1851.
Mazariegos, G. V., Morton, D. H., Sindhi, R., Soltys, K., Nayyar, N., Bond, G., Shellmer, D., Shneider, B., Vockley, J., and Strauss, K. A. (2012). Liver transplantation for classical maple syrup urine disease: long-term follow-up in 37 patients and comparative United Network for Organ Sharing experience. J. Pediatr. 160, 116-121.e1.
Muelly, E. R., Moore, G. J., Bunce, S. C., Mack, J., Bigler, D. C., Morton, D. H., and Strauss, K. A. (2013). Biochemical correlates of neuropsychiatric illness in maple syrup urine disease. J. Clin. Invest. 123, 1809-1820.
Nathwani, A. C., Reiss, U. M., Tuddenham, E. G. D., Rosales, C., Chowdary, P., McIntosh, J., Della Peruta, M., Lheriteau, E., Patel, N., Raj, D., et al. (2014). Long-term safety and efficacy of factor IX gene therapy in hemophilia B. N. Engl. J. Med. 371, 1994-2004.
S Sonnet, D., N O'Leary, M., A Gutierrez, M., M Nguyen, S., Mateen, S., Hsu, Y., P Mitchell, K., J Lopez, A., Vockley, J., K Kennedy, B., et al. (2016). Metformin inhibits Branched Chain Amino Acid (BCAA) derived ketoacidosis and promotes metabolic homeostasis in MSUD. Sci. Rep. 6, 28775.
Strauss, K. A., Puffenberger, E. G., and Morton, D. H. (2013). Maple Syrup Urine Disease. In GeneReviews®, M. P. Adam, H. H. Ardinger, R. A. Pagon, S. E. Wallace, L. J. Bean, K. Stephens, and A. Amemiya, eds. (Seattle (Wash.): University of Washington, Seattle), p.
Suryawan, A., Hawes, J. W., Harris, R. A., Shimomura, Y., Jenkins, A. E., and Hutson, S. M. (1998). A molecular model of human branched-chain amino acid metabolism. Am. J. Clin. Nutr. 68, 72-81.
Wendel, U., Saudubray, J. M., Bodner, A., and Schadewaldt, P. (1999). Liver transplantation in maple syrup urine disease. Eur. J. Pediatr. 158 Suppl 2, S60-64.
Yoneshiro, T., Wang, Q., Tajima, K., Matsushita, M., Maki, H., Igarashi, K., Dai, Z., White, P. J., McGarrah, R. W., Ilkayeva, O. R., et al. (2019). BCAA catabolism in brown fat controls energy homeostasis through SLC25A44. Nature 572, 614-619.
Zinnanti, W. J., Lazovic, J., Griffin, K., Skvorak, K. J., Paul, H. S., Homanics, G. E., Bewley, M. C., Cheng, K. C., Lanoue, K. F., and Flanagan, J. M. (2009). Dual mechanism of brain injury and novel treatment strategy in maple syrup urine disease. Brain J. Neurol. 132, 903-918.

1. A recombinant nucleic acid molecule comprising a transgene encoding for the branched-chain keto acid decarboxylase alpha or beta subunit wherein the transgene is operatively linked to a promoter.

2. The recombinant nucleic acid molecule of claim 1 wherein the transgene comprises a nucleic acid sequence having at least 80% of identity with SEQ ID NO:1 or SEQ ID NO:2.

3. The recombinant nucleic acid molecule of claim 1 wherein the sequence of the transgene is codon-optimized

4. The recombinant nucleic acid molecule of claim 3 wherein the transgene comprises the nucleic acid sequence of SEQ ID NO:3, SEQ ID NO:4, SEQ ID NO:5 or SEQ ID NO:6.

5. The recombinant nucleic acid molecule of claim 1 wherein the promoter is selected to drive the expression of the transgene specifically in the liver.

6. The recombinant nucleic acid molecule of claim 5 wherein the promoter is the hAAT promoter that comprises the nucleic acid sequence of SEQ ID NO:7.

7. The recombinant nucleic acid molecule of claim 1 wherein the promoter is selected to drive the expression of the transgene not specifically in the liver.

8. The recombinant nucleic acid molecule of claim 7 wherein the promoter is the EF1a promoter that comprises the nucleic acid sequence of SEQ ID NO:8.

9. The recombinant nucleic acid molecule of claim 7 wherein the EF1a promoter further comprises an extra intronic sequence that will increase the expression of the transgene by the promoter.

10. The recombinant nucleic acid molecule of claim 9 wherein the extra intronic sequence consists of the nucleic acid sequence of SEQ ID NO:9.

11. The recombinant nucleic acid molecule of claim 1 comprising the Woodchuck Hepatitis Virus (WHP) Posttranscriptional Regulatory Element (WPRE) sequence of SEQ ID NO:10.

12. The recombinant nucleic acid molecule of claim 1 comprising a polyadenylation signal sequence inserted downstream to the transgene of SEQ ID NO:11.

13. The recombinant nucleic acid molecule of claim 1 comprising the inverted terminal repeats (ITRs) sequences of SEQ ID NO:12.

14. The recombinant nucleic acid molecule of claim 1 comprising a nucleic acid sequence selected from the group consisting of SEQ ID NO:13 to SEQ ID NO:24.

15. A recombinant AAV8 viral particle that comprises the recombinant nucleic acid molecule of claim 1.

16. A method of treating maple syrup urine disease in a subject in need thereof comprising administering to the subject a therapeutically effective amount of the recombinant AAV8 viral particle of claim 15.

17. The method of claim 16 wherein the recombinant AAV8 viral particle is administered to the subject intravenously.
